(19) United States
(12) Patent Application Publication
(10) Pub. No.: US 2013/0144322 A1
(43) Pub. Date: Jun. 6, 2013

(54) DEVICES AND METHODS TO TREAT GALLSTONE DISEASE

(71) Applicants: Matthew John Callaghan, Palo Alto, CA (US); Kevin Z. Chao, Palo Alto, CA (US); John Avi Roop, Palo Alto, CA (US); Joelle Abra Faulkner, Palo Alto, CA (US)

(72) Inventors: Matthew John Callaghan, Palo Alto, CA (US); Kevin Z. Chao, Palo Alto, CA (US); John Avi Roop, Palo Alto, CA (US); Joelle Abra Faulkner, Palo Alto, CA (US)

(21) Appl. No.: 13/696,810

(22) PCT Filed: May 9, 2011
(86) PCT No.: PCT/US11/35797

§ 371 Date: Feb. 20, 2013

Related U.S. Application Data
(60) Provisional application No. 61/332,764, filed on May 8, 2010.

(51) Int. Cl. A61F 2/04

(52) U.S. Cl. CPC A61F 2/04
USPC 606/191

(57) ABSTRACT

A number of gallbladder treatment devices and methods treating gallstone disease are included. A gallbladder treatment device has a structure secured within the gallbladder to maintain gallstones and gallstone fragments within the gallbladder interior and distal to the gallbladder neck. A gallbladder treatment device may be put into therapeutic use by first accessing the gallbladder interior and then advancing the gallbladder treatment device into the gallbladder interior. The gallbladder treatment device is then positioned within the gallbladder interior in an orientation to maintain gallstones and gallstone fragments on one side of the device and away from or distal to the gallbladder neck.

### CROSS REFERENCE TO RELATED APPLICATIONS

This application claims the benefit under 35 U.S.C. 119 of U.S. Patent Application No. 61/332,764, filed May 8, 2010, titled “A DEVICE TO TREAT GALLSTONE DISEASE”. This application is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

Embodiments of the present invention relate to devices and methods of treatment of the gallbladder by maintaining gallstones or gallstone fragments on one side of device and away from or distal to the gallbladder neck. It is believed and that the devices and methods described herein may be used to prevent or mitigate gallbladder obstruction or impaction due to gallstones.

### BACKGROUND OF THE INVENTION

Gallstone disease affects up to 20 million people in the United States and carries a direct healthcare cost of more than $6.3 billion per year. See Strasberg, S. M., Clinical practice. Acute calculous cholecystitis. N Engl J Med, 2008. 358(26): p. 2804-11. While most patients are asymptomatic, biliary colic develops in up to 5% annually. Gallstones are more common among women, with 25% of women having gallstones by the age of 60 and 50% by the age of 75. See Wilund, K. R., et al., Endurance exercise training reduces gallstone development in mice. J Appl Physiol, 2008. 104(3): p. 761-5. Other risk factors include pregnancy, hormone replacement therapy, obesity, rapid weight loss, diabetes, Crohn's disease, family history, older age, spinal cord injury, prolonged fasting, total parenteral nutrition, and somatostatin analogue treatment. See Machado, N. O. and L. S. Machado, Laparoscopic cholecystectomy in the third trimester of pregnancy: report of 3 cases. Surg Laparosc Endosc Percutan Tech, 2009. 19(6): p. 439-41. Outside the US, the rate of gallstone disease has been climbing rapidly and now exceeds 80 million. See Marschall, H. U. and C. Einarsson, Gallstone disease. J Intern Med, 2007. 261(6): p. 529-42. This is attributed primarily to an increase in dietary cholesterol, which also increases the risk of obesity, diabetes and heart failure.

Gallstones are most commonly formed from cholesterol, but may also be made of calcium bilirubinate, in which case they care referred to as pigment stones. Pigment stones are more common in Asia and Africa. See Marschall, H. U. and C. Einarsson, Gallstone disease. J Intern Med, 2007. 261(6): p. 529-42. Stones range in size from a few millimeters to several centimeters. They can form in the gallbladder, cystic duct, or common bile duct, although the most common location for formation is within the fundus and body of the gallbladder. Stones themselves do not necessarily result in disease states. However, when a stone obstructs the outlet of the gallbladder during contraction, bile flow is restricted which can result in infection and inflammation. It is estimated that stones <3 mm can pass through the gallbladder and duct network into the intestines without complication. Stones >3 mm pose a risk of impaction or obstruction.

Factors that are thought to contribute to the complex process of gallstone formation are supersaturation of bile with cholesterol and gallbladder dysmotility. Cholesterol content of bile is determined by not only diet but the absorption and secretion processes of the gallbladder epithelium. The gallbladder can absorb water, electrolytes, acidify the bile and secrete mucous glycoproteins. It is thought that the sequence of events in the formation of gallstones are supersaturation of bile, nucleation, precipitation, and growth of microcrystals to gallstones. Oldham-Ott, C. K. and J. Gilloteaux, Comparative morphology of the gallbladder and biliary tract in vertebrates: variation in structure, homology in function and gallstones. Microsc Res Tech, 1997. 38(6): p. 571-97. Bile salts and lecithin are important for keeping cholesterol solubilized in bile. There is a critical balance between pronucleating and antinucleating factors in bile that prevent or permit gallstone formation. It has been thought that mucin serves as a nucleus for stone formation because its peptide core contains hydrophobic domains that have a high affinity for lipids. However, ˜50% of the population in developed countries have bile supersaturation and only ˜10% of this population develops gallstones. It has also been noted that gallbladder evacuation is slower and less complete in gallstone patients. Gallbladder dysmotility may predispose to gallstones and may be worsened by the presence of gallstones. It is thought that supersaturated bile may alter the lipid composition of the gallbladder smooth muscle cells and thus their function.

Chronic symptomatic gallstone disease is called biliary colic and is the manifestation of repeated but temporary obstruction of the cystic duct by gallstones. Symptoms include pain in the upper right quadrant, nausea, and/or vomiting. Fever and systemic symptoms are unusual and may indicate the development of acute cholecystitis. The pain may resolve within a hour of onset, as the stones fall back from the cystic duct outlet when gallbladder contraction ceases. Recurrence is common and more likely after ingestion of a fatty or large meal.

Acute cholecystitis is a surgical emergency and can develop in 1-3% of those with symptomatic disease. Symptoms are similar to biliary colic, although more severe and persistent and accompanied by fever. Complications from can be life threatening and include gangrene, perforation and abscesses in and around the gallbladder, which can be fatal if not treated.

### Treatment of Gallstone Disease

The most common and effective treatment for gallstone disease today is surgical removal of the gallbladder. In patients of acceptable surgical risk, the gallbladder is removed through either open, or more commonly, laparoscopic surgery. Approximately 80% of gallbladder surgeries are performed laparoscopically and approximately 800,000 of these surgeries are performed each year in the US. See Sanders, G. and A. N. Kingsnorth, Gallstones. BMJ, 2007. 335(7614): p. 295-9. Laparoscopic surgery carries a 1-2% risk of a severe complication such as common bile duct transaction and an overall (total) complication risk of 26.6%. Keus, F., et al., Laparoscopic versus open cholecystectomy for patients with symptomatic cholecystolithiasis. Cochrane Database Syst Rev, 2006(4): p. CD006231. Risks are increased in older, obese, and diabetic patients and those with cardiac or pulmonary conditions. Approximately 25% of patients with symptomatic gallstone disease or cholecystitis are over the age of 65 and carry a higher risk for surgery. Lammert, F. and J. F. Miguel, Gallstone disease: from genes to evidence-based therapy. J Hepatol, 2008. 48 Suppl 1: p. S124-35. The conversion rate from laparoscopic to open surgery is 13% and the re-operation rate after laparoscopic cholecystectomy is 1.6%. See Kirshtein, B., et al., Laparoscopic cholecystectomy for acute cholecystitis in the elderly: is it safe? Surg Laparosc Endosc Percutan Tech, 2008. 18(4): p. 334-9. The hospital stay for laparoscopic patients is 1.1 days, but can be as long as 5-7 days for open or complicated procedures. See Annamaneni, R. K., D. Moraitis, and C. G. Cayten, Laparoscopic cholecystectomy in the elderly. JSLS, 2005. 9(4): p. 408-10. Repair of cholecystectomy-related bile duct injuries can run 4.5 to 26 times the cost of an uncomplicated procedure and carries a significant mortality rate. See Savader, S. J., et al., Laparoscopic cholecystectomy-related bile duct injuries: a health and financial disaster. Ann Surg, 1997. 225(3): p. 268-73.

In cases where the surgical risk is too high, acute patients are referred for drainage through a percutaneous catheter in combination with intravenous antibiotics. Placement of a drainage tube is called cholecystostomy and is performed using ultrasound and fluoroscopic guidance. The drainage catheter is usually left in place for up to 6 weeks.

Non-acute patients who are not surgical candidates can be referred for medical management. Non-surgical treatment modalities include observation or expectant management, dissolution therapy, and extracorporeal shock wave lithotripsy. Oral dissolution consists of daily administration of bile acids to gradually dissolve gallstones. Oral therapy is more effective in patient with cholesterol stones and the rate of recurrence after treatment is high.

Lithotripsy involves the application of high frequency ultrasound waves to break stones into smaller fragments, which can then pass through the cystic duct. Treatment is expensive and recurrence rates are high, reaching 80% at 10 years after treatment. See Paumgartner, G. and G. H. Sauter, Extracorporeal shock wave lithotripsy of gallstones: 20th anniversary of the first treatment. Eur J Gastroenterol Hepatol, 2005. 17(5): p. 525-7. Unfortunately, the size of the fragments created by lithotripsy cannot be accurately controlled and may result in smaller stones more likely to impact within the cystic or common bile ducts.

For stones which have passed out of the gallbladder and impacted within the distal biliary tree, endoscopic retrograde cholangiopancreatogrpahy (ERCP) can be performed to retrieve these stones and restore patency to the biliary system. However, ERCP cannot retrieve stones within the gallbladder and does not prevent future recurrences. Complications rates for ERPC approach 8-10% and include pancreatitis, infection, bleeding, and perforation.

### Gallstones and Bariatric Surgery

An obesity epidemic in the United States has resulted in exponential growth in the number of bariatric procedures performed annually. Regardless of procedure type, patients undergoing weight loss surgery are at increased risk for gallstone formation and thus subsequent stone impaction during the first 12 months after their surgery. See Wudel, L. J., Jr., et al., Prevention of gallstone formation in morbidly obese patients undergoing rapid weight loss: results of a randomized controlled pilot study. J Surg Res, 2002. 102(1): p. 50-6. Cholesterol stored in peripheral tissues is mobilized during rapid weight loss and concentrated in bile, where it can precipitate and form gallstones. As body weight stabilizes, the risk of stone formation returns to baseline. Patients with symptomatic gallstones during this early post-operative period pose a higher surgical risk due to their weight, liver size and intra-abdominal adhesions formed after their procedure. Once they have lost weight and recovered from surgery, their operative risk is decreased and it is generally considered safe to perform a cholecystectomy if indicated. Thus, it would be beneficial to provide a temporary means to prevent symptomatic cholelithiasis or the development of acute cholecystitis until body weight is reduced and stabilized.

### Preventing the Passage of Potentially Obstructive Gallstones

Because only stones of a certain size, for example greater than 3 mm in diameter, are at risk for obstruction and impaction, it is desirable to prevent these stones from passing out of the gallbladder. What is needed are devices and methods for maintaining gallstones and/or gallstone fragments within the gallbladder and distal to the gallbladder neck in order to aid in the prevention or mitigation of gallbladder obstruction or impaction.

### SUMMARY OF THE INVENTION

There are provided herein a number of alternative gallbladder treatment devices for maintaining a gallstone or a gallstone fragment within a portion of a gallbladder distal to a gallbladder neck. In one aspect, the device includes a frame formed from at least one element having a filtering characteristic dimensioned to prevent the passage of a gallstone or gallstone fragment having a largest dimension of more than 3 mm. There is also a plurality of struts extending from the frame to secure the frame in an orientation transverse to a gallbladder central, longitudinal axis. As a result, the gallstone or the gallstone fragments having a largest dimension of more than 3 mm remains within the gallbladder on one side of the frame. In one variation, the frame has a generally rounded contour with a radius of curvature between about 0.1 cm to about 1.0 cm. In another variation, the frame has a generally conical contour with a radius of curvature between about 0.1 cm to about 1.0 cm.

In one embodiment, the at least one element is a flexible sheet of material and the filtering characteristic is a plurality of apertures formed in the flexible sheet having a largest dimension of less than 3 mm. In another variation, the at least one element is a plurality of flexible strands from a braided structure that maintains a filtering characteristic that is the spacing between adjacent strands that is less than 3 mm. Still further, in one embodiment, the at least one element is a plurality of suture strands attached to the frame that maintains a filtering characteristic that is the spacing between adjacent suture strands that is less than 3 mm.

In one aspect, the one or more fixation elements are generally evenly spaced around the perimeter. The one or more fixation elements may include a hook, a ring, a pivoting barbed tip, or a strand of suture, alone or in any combination. In addition, the fixation elements describe herein may have a length selected to penetrate only partially through one or more layers of a gallbladder wall or completely through all layers of a gallbladder wall.

In one variation, a portion of the gallbladder treatment device struts include a fixation element to engage with a wall of the gallbladder. In one variation, the fixation element is a plurality of spikes. In other gallbladder treatment devices, a portion of each strut has an opening to permit passage of a fixation element. A fixation element may be sized to pass through the opening having a length at least partially or completely penetrate through a gallbladder wall.

In some aspects, the fixation elements of a gallbladder treatment include one or more penetration elements that are supported by a wall contacting section. The fixation elements or penetration elements has a length to pierce through substantially all layers of a gallbladder wall. Optionally, each of the fixation or penetration elements has a length to remain within without piercing through a gallbladder wall. Fixation elements or penetration elements may be in a variety of forms such as a barb, a spike or a needle. In some aspects, one or more fixation or penetration elements are attached to or formed on the surface of the wall contacting section. Still further, such elements may take the form of a separate nail or a separate tack that passes through a corresponding opening in the wall contacting section.

In some variations, all or a portion of one or more components of a gallbladder treatment device has a surface treatment and/or coating to encourage fibrous in-growth and/or inhibit gallbladder biofilm formation. In another aspect, a portion of the wall contacting portion of a gallbladder treatment device is treated for in-growth. In one variation, a portion of the wall contacting portion is roughened to promote in-growth. In one aspect, a portion of the wall contacting portion includes spikes or barbs to promote in-growth. In one variation, a fixation element that is a portion of the gallbladder treatment device strut and/or the strut itself is coated or treated to encourage fibrous in-growth. In one aspect, the surface treatment is a coating of a growth promoting compound. The growth promoting compound comprises polyethyleneglycol, in one aspect. In another aspect, the growth promoting compound comprises an epithelial growth factor. The surface treatment is a texturing to promote in-growth in some embodiments. In some other variations, at least a portion of the frame or a portion of the struts is coated with a material to inhibit gallbladder biofilm formation. The material is an antibiotic selected from the group of antibiotics suited to treat infections of the gastrointestinal tract, in some aspects. The antibiotic may be a ciprofloxacin.

There are also gallbladder treatment devices having at least a portion of the frame or a portion of the struts that are formed from materials having substantially non-porous, hydrophillic surfaces. Additionally or alternatively, the plurality of struts are shaped to deflect outwardly against the gallbladder wall with a radial force sufficient to maintain the frame in the orientation transverse to a gallbladder central, longitudinal axis. In some aspects, the gallbladder treatment device maintains a transverse orientation when the gallbladder is in a contracted condition and an expanded condition.

In some aspects, the plurality of struts in a gallbladder treatment device are shaped to conform to a portion of the curvature of a wall of the gallbladder fundus so as to maintain the frame in a portion of the gallbladder fundus in an orientation transverse to a gallbladder central, longitudinal axis in order to maintain the gallstone or the gallstone fragment having a largest dimension of greater than 3 mm on one side of the frame and within a more distal portion of the gallbladder fundus.

In some aspects, the plurality of struts in a gallbladder treatment device are shaped to conform to a portion of the curvature of a wall of the gallbladder body so as to maintain the frame in portion of the gallbladder body in an orientation transverse to a gallbladder central, longitudinal axis in order to maintain the gallstone or the gallstone fragment having a largest dimension of greater than 3 mm on one side of the frame and within a portion of the gallbladder fundus and a more distal portion of the gallbladder body.

In some aspects, the plurality of struts in a gallbladder treatment device are shaped to conform to a portion of the curvature of a wall of the gallbladder infundibulum so as to maintain the frame in a portion of the gallbladder infundibulum in an orientation transverse to a gallbladder central, longitudinal axis in order to maintain the gallstone or the gallstone fragment having a largest dimension of greater than 3 mm on one side of the frame and within a portion of the gallbladder fundus, the gallbladder body and a more distal portion of the gallbladder infundibulum.

In some aspects, the plurality of struts in a gallbladder treatment device are shaped to conform to a portion of the curvature of a wall of the gallbladder neck so as to maintain the frame in a portion of the gallbladder neck in an orientation transverse to a gallbladder central, longitudinal axis in order to maintain the gallstone or the gallstone fragment having a largest dimension of greater than 3 mm on one side of the frame and within a portion of the gallbladder fundus, the gallbladder body and the gallbladder infundibulum.

In some embodiments or alternatives of the above, the plurality of struts in a gallbladder treatment device are shaped into a closed or substantially closed three dimensional structure. In some aspects, the closed or substantially closed three dimensional structure is generally shaped as a sphere or an ovoid that is dimensioned to fit within the gallbladder interior.

In another embodiment, there is provided a gallbladder treatment device for maintaining a gallstone or a gallstone fragment within a portion of a gallbladder distal to a gallbladder neck. In this embodiment, there are a plurality of struts arranged in a generally cylindrical pattern and a wall contacting section on each of the plurality of struts. There is also a frame formed from a plurality of flexible members attached between two of the plurality of struts wherein the spacing between two adjacent flexible members when attached between two of the plurality of struts is less than 3 mm such that in use in the gallbladder the frame maintains the gallstone or the gallstone fragment within a portion of a gallbladder distal to a gallbladder neck. In addition, the radius of curvature formed by the plurality of flexible members is variable between a first curvature when the plurality of struts are contacting an expanded gallbladder and a second curvature when the plurality of struts are contacting a contracted gallbladder. In one aspect, a filter material is supported on the frame.

In another gallbladder treatment device alternative, the device includes a plurality of struts having a first end and a second end. A curved portion between the first end and the second end is moveable between a first condition having a radius of curvature induced by a contracted gallbladder and a second condition having a radius of curvature induced by an expanded gallbladder. The plurality of struts overlap one another at least once between the first end and the second end. The resulting pattern of overlapping struts prevents the passage of a gallstone or gallstone fragment greater than 3 mm.

In still another alternative of a gallbladder treatment device for maintaining a gallstone or a gallstone fragment within a portion of a gallbladder distal to a gallbladder neck, the device includes a structure formed from at least one element having a filtering characteristic dimensioned to prevent the passage of a gallstone or gallstone fragment having a largest dimension of greater than 3 mm. There is also one or more fixation elements spaced along and attached to the perimeter of the structure.

In one aspect, the filtering structure comprises a braided structure, or a mesh filter of intersection strands. The strands may be made from a shape memory metal or be strands of a suture material. The structure may be a flexible sheet in one aspect. When formed from a sheet, the filtering characteristic dimensioned to prevent the passage of a gallstone or gallstone fragment having a largest dimension of greater than 3 mm are a plurality of perforations formed in the flexible sheet.

In another aspect of a gallbladder treatment device, the structure formed from at least one element having a filtering characteristic dimensioned to prevent gallstone passage is dimensioned to span a portion of the gallbladder transverse to the gallbladder central longitudinal axis. The portion of the gallbladder transverse to the gallbladder central longitudinal axis includes, for example, a portion of the gallbladder fundus; a portion of the gallbladder body; a portion of the gallbladder infundibulum; and a portion of the gallbladder neck.

There are also provided herein a number of methods for maintaining a gallstone or a gallstone fragment within a portion of a gallbladder distal to a gallbladder neck. In some embodiments, the methods include accessing the interior of the gallbladder with a delivery device. Next, there is the step of advancing a gallbladder treatment device into the gallbladder interior using the delivery device. Thereafter, the gallbladder treatment device is positioned within the gallbladder to maintain a gallstone or a gallstone fragment within a portion of the gallbladder distal to the gallbladder neck and one side of the gallbladder treatment device.

Accessing the interior of the gallbladder may be accomplished with an open surgical procedure or one of a variety of minimally invasive surgical (M.I.S.) procedures.

The minimally invasive surgical procedure includes, for example, accessing the interior of the gallbladder with an endoscopic or a laparoscopic approach technique. Moreover, the minimally invasive surgical procedure includes, for example, accessing the interior of the gallbladder with a percutaneous or a transluminal approach technique. In addition, both open and M.I.S. procedures may include accessing the interior of the gallbladder with a transhepatic or a transperitoneal approach technique.

In another aspect of the inventive methods, the advancing step includes, for example, passing the gallbladder treatment device in a stowed condition through at least a portion of a liver including, for example, a portion of the liver lacking a peritoneal covering. In still another aspect of the inventive methods, the advancing step includes, for example, passing the gallbladder treatment device in a stowed condition through at least a portion of a peritoneal cavity or through at least a portion of the gastrointestinal tract. Still further, in some aspects of passing the gallbladder treatment device through at least a portion of the gastrointestinal tract the stowed gallbladder treatment device is passed though a portion of an esophagus, a stomach, a duodenum, a small intestine, or a colon. In still other aspects, alternatives of the advancing step include, for example, passing the gallbladder treatment device through a wall of the gastrointestinal tract. In still other embodiments, the step of passing through a wall of the gastrointestinal tract includes a wall in a portion of an esophagus, a stomach, a duodenum, a small intestine, or a colon.

In still other aspects of the inventive methods, the advancing step includes, for example, passing the gallbladder treatment device in a stowed condition through a portion of a fundus of the gallbladder; through a portion of a body of the gallbladder; through a portion of an infundibulum of the gallbladder; through an generally inferior aspect of the gallbladder; through a generally superior aspect of the gallbladder; and/or through a generally lateral aspect of the gallbladder.

In still additional aspects, the positioning step includes, for example, adding and/or removing fluid from the gallbladder. In another aspect, the positioning step includes, for example, activating a delivery device. In one version, activating a delivery device includes inflating a balloon. In another variation, the positioning step includes, for example, moving or transitioning the gallbladder treatment device from a stowed configuration to a deployed configuration. Still further, the positioning step may include adjusting the orientation of the gallbladder treatment device within the gallbladder.

In one aspect, after the positioning step, the gallbladder treatment device is positioned within the gallbladder in an orientation that retains a portion the gallstones and stone fragments in the gallbladder substantially within a fundus of the gallbladder. In another variation, after the positioning step, the gallbladder treatment device is positioned within the gallbladder in an orientation that retains a portion the gallstones and stone fragments in the gallbladder substantially within a fundus and a body of the gallbladder. In another variation, after the positioning step, the gallbladder treatment device is positioned within the gallbladder in an orientation that retains a portion the gallstones and stone fragments in the gallbladder substantially within a fundus, a body and a portion of an infundibulum of the gallbladder. Moreover, it is appreciated that, after the positioning step, the gallbladder treatment device is against a wall of a fundus of the gallbladder; against a wall of a body of the gallbladder; against a wall of an infundibulum of the gallbladder or against a less elastic portion of a wall of an infundibulum of the gallbladder.

The various methods described above are subject to further alternatives. Any of the methods above may include the additional step of anchoring the gallbladder treatment device to the gallbladder wall or a portion thereof. Still further, the methods may also include the step inflating the gallbladder after the accessing step and before the advancing and positioning steps. Similarly, any of the methods above may also include collapsing the gallbladder around the gallbladder delivery device after the advancing or the positioning steps. Collapsing may be accomplished using suction from a catheter delivery device, endoscope, or other acceptable surgical device and suction procedure.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings in which

In the drawings:

FIG. 1A illustrates the gallbladder within the abdomen and in relation to the liver with a section of the gallbladder wall removed to show the gallbladder interior volume or lumen having gallstones and gallstone fragments therein;

FIG. 1B is an section of the gallbladder wall showing the relative locations of the various layers with regard to one another and the peritoneum or abdominal wall and the abdominal cavity;

FIGS. 2A and 2B are isometric and enlarged views, respectively, of an embodiment of a gallbladder treatment device;

FIG. 3 illustrates the gallbladder as shown in FIG. 1A with a section of the gallbladder wall removed to show an exemplary gallbladder treatment device within the gallbladder interior positioned in an orientation to maintain the gallstones or gallstone fragments on one side of the device and away from the gallbladder neck;

FIG. 3A is an enlarged section view of a strut of the device in FIG. 3 showing a wall contacting section of the strut along a portion of the gallbladder wall;

FIG. 3B is an enlarged view of a strut of the device in FIG. 3 showing a wall contacting section of the strut with fixation elements used for within the gallbladder wall fixation along a portion of the gallbladder wall such the short spikes or barbs penetrate at least partially into the gallbladder wall;

FIG. 3C is an enlarged section view of a strut of the device in FIG. 3 showing a wall contacting section of the strut with fixation elements used for through the gallbladder wall fixation along a portion of the gallbladder wall such that the clips penetrate completely through the gallbladder wall;

FIG. 3D is an enlarged section view of a strut of the device in FIG. 3 showing a wall contacting section of the strut having an opening to permit a separate fixation element to pass though the opening and then partially or completely through the gallbladder wall;

FIG. 4A illustrates the gallbladder as shown in FIG. 1A with a section of the gallbladder wall removed to show an exemplary gallbladder treatment device with a conical frame contour within the gallbladder interior positioned in an orientation to maintain the gallstones or gallstone fragments on one side of the device and away from the gallbladder neck;

FIG. 4B illustrates the gallbladder as shown in FIG. 1A with a section of the gallbladder wall removed to show an exemplary gallbladder treatment device with a rounded frame contour within the gallbladder interior positioned in an orientation to maintain the gallstones or gallstone fragments on one side of the device and away from the gallbladder neck;

FIG. 5A illustrates the gallbladder as shown in FIG. 1A with a section of the gallbladder wall removed to show an exemplary gallbladder treatment device with a rounded frame contour and struts adjusted to increase the wall contacting section within the gallbladder interior positioned in an orientation to maintain the gallstones or gallstone fragments on one side of the device and away from the gallbladder neck;

FIG. 5B illustrates the gallbladder as shown in FIG. 1A with a section of the gallbladder wall removed to show an exemplary gallbladder treatment device with a rounded frame contour and struts adjusted to decrease the wall contacting section within the gallbladder interior positioned in an orientation to maintain the gallstones or gallstone fragments on one side of the device and away from the gallbladder neck;

FIGS. 6A and 6B illustrate the gallbladder as shown in FIG. 1A in an expanded and contracted condition, respectively, with a section of the gallbladder wall removed to show an exemplary hoop framed gallbladder treatment device shown in FIG. 6C within the gallbladder infundibular interior positioned in an orientation to maintain the gallstones or gallstone fragments on one side of the device and away from the gallbladder neck;

FIGS. 7A and 7B illustrate the gallbladder as shown in FIG. 1A in an expanded and contracted condition, respectively, with a section of the gallbladder wall removed to show an exemplary frameless gallbladder treatment device (here a mesh sphere) the gallbladder fundus interior positioned in an orientation to maintain the gallstones or gallstone fragments on one side of the device and away from the gallbladder neck;

FIG. 8A illustrates an exemplary gallbladder treatment device structure formed from at least one element having a filtering characteristic dimensioned to prevent the passage of a gallstone or gallstone fragment having a largest dimension of greater than 3 mm shown here as a braided structure with loop shaped fixation elements (FIG. 9A) spaced along and attached to the perimeter of the braided structure;

FIG. 8B illustrates an exemplary gallbladder treatment device structure formed from at least one element having a filtering characteristic dimensioned to prevent the passage of a gallstone or gallstone fragment having a largest dimension of greater than 3 mm shown here as a mesh filter of intersecting strands with hook fixation elements (FIG. 9B) spaced along and attached to the perimeter of the filter elements;

FIG. 8C illustrates an exemplary gallbladder treatment device structure formed from at least one element having a filtering characteristic dimensioned to prevent the passage of a gallstone or gallstone fragment having a largest dimension of greater than 3 mm shown here as a perforated flexible sheet with pivoting barbed tip fixation elements (FIG. 9C) spaced along and attached to the perimeter;

FIG. 9A illustrates a loop shaped fixation element as shown with the gallbladder treatment device of FIG. 8A;

FIG. 9B illustrates a hook shaped fixation element as shown with the gallbladder treatment device of FIG. 8B;

FIG. 9C illustrates a pivoting barbed tip fixation element as shown with the gallbladder treatment device of FIG. 8C;

FIGS. 10A and 10B illustrate the gallbladder as shown in FIG. 1A in an expanded and contracted condition, respectively, with a section of the gallbladder wall removed to show an exemplary gallbladder treatment device with a rounded frame contour within the gallbladder body interior positioned in an orientation to maintain the gallstones or gallstone fragments on one side of the device and away from the gallbladder neck;

FIGS. 10C and 10D illustrate the gallbladder as shown in FIG. 1A in an expanded and contracted condition, respectively, with a section of the gallbladder wall removed to show an exemplary gallbladder treatment device with a conical frame contour within the gallbladder body interior positioned in an orientation to maintain the gallstones or gallstone fragments on one side of the device and away from the gallbladder neck;

FIGS. 11A and 11B illustrate the gallbladder as shown in FIG. 1A in an expanded and contracted condition, respectively, with a section of the gallbladder wall removed to show an exemplary gallbladder treatment device with a rounded frame contour within the gallbladder infundibulum interior positioned in an orientation to maintain the gallstones or gallstone fragments on one side of the device and away from the gallbladder neck;

FIGS. 11C and 11D illustrate the gallbladder as shown in FIG. 1A in an expanded and contracted condition, respectively, with a section of the gallbladder wall removed to show an exemplary gallbladder treatment device with a conical frame contour within the gallbladder infundibulum interior positioned in an orientation to maintain the gallstones or gallstone fragments on one side of the device and away from the gallbladder neck;

FIGS. 12A and 12B illustrate the gallbladder as shown in FIG. 1A in an expanded and condition with a section of the gallbladder wall removed to show an exemplary gallbladder treatment device with a conical frame contour within the gallbladder neck or cystic duct entrance positioned in an orientation to maintain the gallstones or gallstone fragments on one side of the device and unable to enter within or block the gallbladder neck or cystic duct;

FIG. 13A illustrates the gallbladder as shown in FIG. 1A with a section of the gallbladder wall removed to show an exemplary gallbladder treatment device in a stowed condition at the distal end of a delivery device or catheter that has been passed through an opening in the gallbladder wall and into the gallbladder interior;

FIG. 13B illustrates the gallbladder and treatment device as shown in FIG. 13A undergoing a balloon dilation procedure to position the gallbladder treatment device within the gallbladder body in an orientation to maintain the gallstones or gallstone fragments on one side of the device and away from the gallbladder neck;

FIG. 14 is a block diagram illustrating various methods of accessing the interior of the gallbladder, advancing a gallbladder treatment device into the gallbladder interior and positioning the gallbladder treatment device within the gallbladder interior in an orientation to maintain the gallstones or gallstone fragments on one side of the device and away from or distal to the gallbladder neck;

FIG. 15A illustrates the gallbladder within the abdomen and in relation to the liver, the pancreas and the duodenum with a section of the gallbladder wall removed to show accessing the gallbladder interior volume with a guide wire and a delivery catheter using a transhepatic approach;

FIG. 15B illustrates the gallbladder within the abdomen and in relation to the liver, the pancreas and the duodenum with a section of the gallbladder wall removed to show accessing the gallbladder interior volume with a guide wire and a delivery catheter using a transperitoneal approach;

FIG. 15C illustrates the gallbladder within the abdomen and in relation to the liver, the pancreas and the duodenum showing accessing the gallbladder endoscopically by advancing an endoscope through an opening in the duodenum and advancing towards the gallbladder; and

FIG. 15D illustrates the gallbladder within the abdomen and in relation to the liver, the pancreas and the duodenum showing accessing the gallbladder endoscopically by advancing an endoscope into the duodenum and then advancing a guidewire and cholangioscope into the common bile duct and advancing towards the gallbladder;

### DETAILED DESCRIPTION OF THE INVENTION

Certain specific details are set forth in the following description and figures to provide an understanding of various embodiments of the invention. Certain well-known details, associated electronics and devices are not set forth in the following disclosure to avoid unnecessarily obscuring the various embodiments of the invention. Further, those of ordinary skill in the relevant art will understand that they can practice other embodiments of the invention without one or more of the details described below. Finally, while various processes are described with reference to steps and sequences in the following disclosure, the description is for providing a clear implementation of particular embodiments of the invention, and the steps and sequences of steps should not be taken as required to practice this invention.

Embodiments of the present invention are directed toward various gall bladder treatment devices and methods of placing such devices within the gallbladder to maintain gallstones and gallstone fragments within the gallbladder and distal to the gallbladder neck. It is believed that the devices and methods described herein may: prohibit or reduce the likelihood of one or more of: (a) transient obstructions of the gallbladder outlet; (b) impaction of the gallbladder outlet; (c) impaction along the cystic duct or common bile duct; and (d) passing of a large gallstone through the gallbladder neck, cystic duct or common bile duct. The effectiveness of the gallbladder treatment devices and placement methods relates at least in part to the segregation of the gallbladder into two regions. One region contains gallstones or gallstone fragments too large to safely pass through and along the gallbladder neck and ducts. The other region contains gallstones or gallstone fragments small enough to safely pass though and along the gallbladder neck and ducts without impairing bile flow or gallbladder function.

Put another way, the gallbladder treatment devices described herein act as a bile filter. In this way, bile within the gallbladder interior 11 passes though the gallbladder neck 18 while gallstones and gallstone fragments 9 that may block or impair the passage of bile as discussed above remain within the gallbladder lumen or volume 11. As such, embodiments of the gallbladder treatment devices may be described as bile filters.

In one aspect, the gallbladder treatment device is sized and positioned within the gallbladder to maintain gallstones and gallstone fragments with a largest dimension of more than 3 mm within the gallbladder and distal to the gallbladder neck. In another aspect, the gallbladder treatment device is sized and positioned within the gallbladder to maintain gallstones and gallstone fragments with a largest dimension of more than 2 mm within the gallbladder and distal to the gallbladder neck. In still another aspect, the gallbladder treatment device is sized and positioned within the gallbladder to maintain gallstones and gallstone fragments with a largest dimension of more than 1 mm within the gallbladder and distal to the gallbladder neck.

In some embodiments, the gallbladder device is maintained in position within the gallbladder by radial force, by one or more fixation devices or by various combinations of radial force and fixation. In one aspect, a gallbladder treatment device may be suited in place within the gallbladder and in a position to maintain gallstones or gallstone fragments distal to the gallbladder neck. In one alternative embodiment, the gallbladder treatment device is a barrier formed from one or more strands of suture stitched across the gallbladder to maintain gallstones and gallstone fragments within the gallbladder while permitting bile to flow through the device and out of the gallbladder neck. In all aspects of the gallbladder device, bile is permitted to flow through or around the device while gallstones or gallstone fragments are maintained on one side of the gallbladder device.

Stone passage can be prevented at multiple points within the gallbladder, according to anatomic location. Sites of intervention include the fundus 12, body 14, infundibulum 16, neck 18, and cystic duct outlet 26. More specifically, the gallbladder treatment device may be positioned within the gallbladder 10 in an orientation that retains a portion the gallstones and stone fragments 9 in the gallbladder substantially within a fundus 12 of the gallbladder; substantially within a fundus 12 and a body 14 of the gallbladder; or in the gallbladder substantially within a fundus 12, a body 14 and a portion of an infundibulum 16 of the gallbladder.

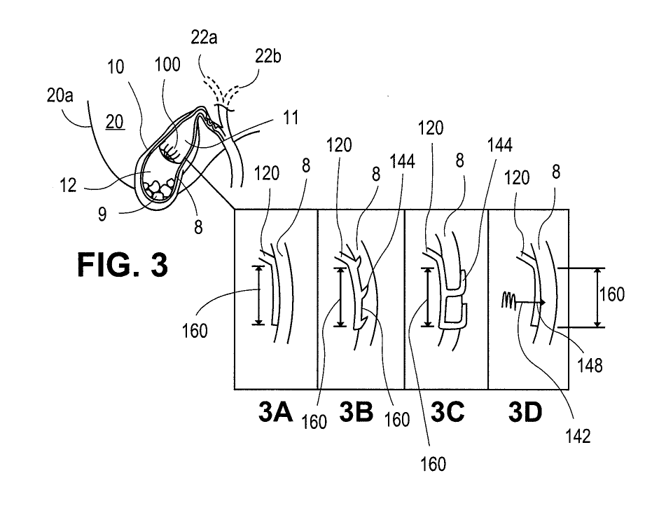

In one aspect, the gallbladder treatment device is maintained by radial force from the device against a gallbladder wall 8 alone or in combination with the use of any of the various fixation elements (e.g., those shown and described in FIGS. 3A-3D and 8A-9C) or surgical fastening techniques (e.g., stapling, suturing and the like). Additionally or alternatively, the gallbladder treatment device may demonstrate only slight or negligible radial force exertion against a gallbladder wall 8. In some instances, the radial force of the device is insufficient alone to maintain the device in place within the gallbladder volume 11. In this case, the fixation element or elements, fixation technique, resulting in-growth or combinations thereof become the primary mode of securing the gallbladder treatment device within the gallbladder lumen or interior volume 11.

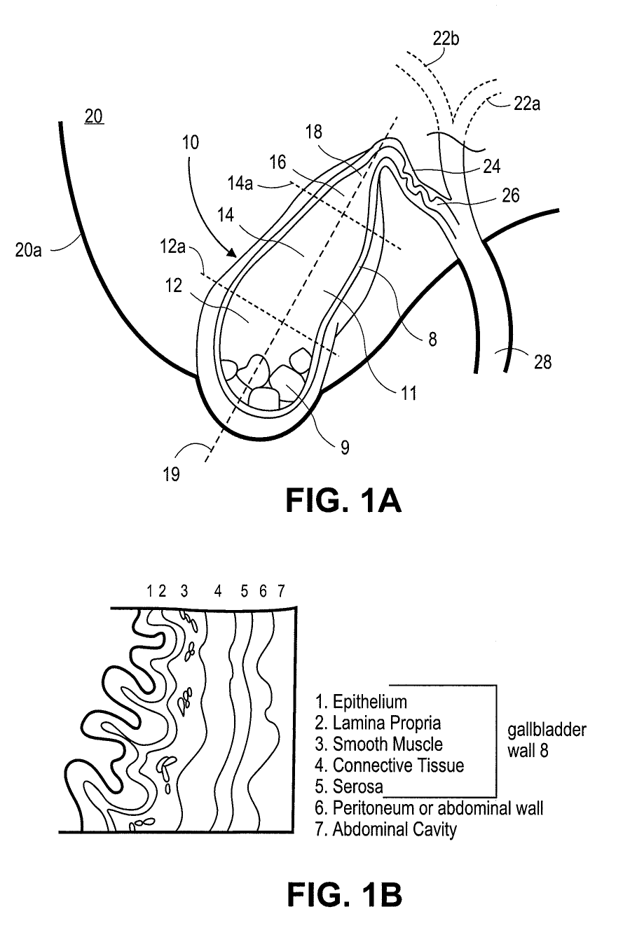

Irrespective of the methods or elements used to maintain the placement, position or orientation of the gallbladder treatment device within the gallbladder lumen 11, the gallbladder treatment device is maintained in an orientation within the lumen 11 and in relation to neck 18 to maintain the gallstones/fragments 9 within the gallbladder lumen 11 and away from the neck 18. In one aspect, this orientation is considered as transverse to the gallbladder central longitudinal axis 19 (see FIG. 1A). Dashed lines 12a and 14a represent basic divisions of the gallbladder lumen 11 between the fundus 12 and body 14 (dashed line 12a) and the body 14 and the infunibulum 16 (dashed line 14a). In addition, the dashed lines 12a and 14a also illustrate an orientation that is transverse to the gallbladder central longitudinal axis 19. Moreover, the angle between the gallbladder central longitudinal axis 19 and the gallbladder treatment device may vary from a generally orthogonal relationship or about 90 degrees as indicated by dashed lines 12a and 14a to any of a wide variety of angles so long as the gallbladder device retains gallstones within the gallbladder lumen 11.

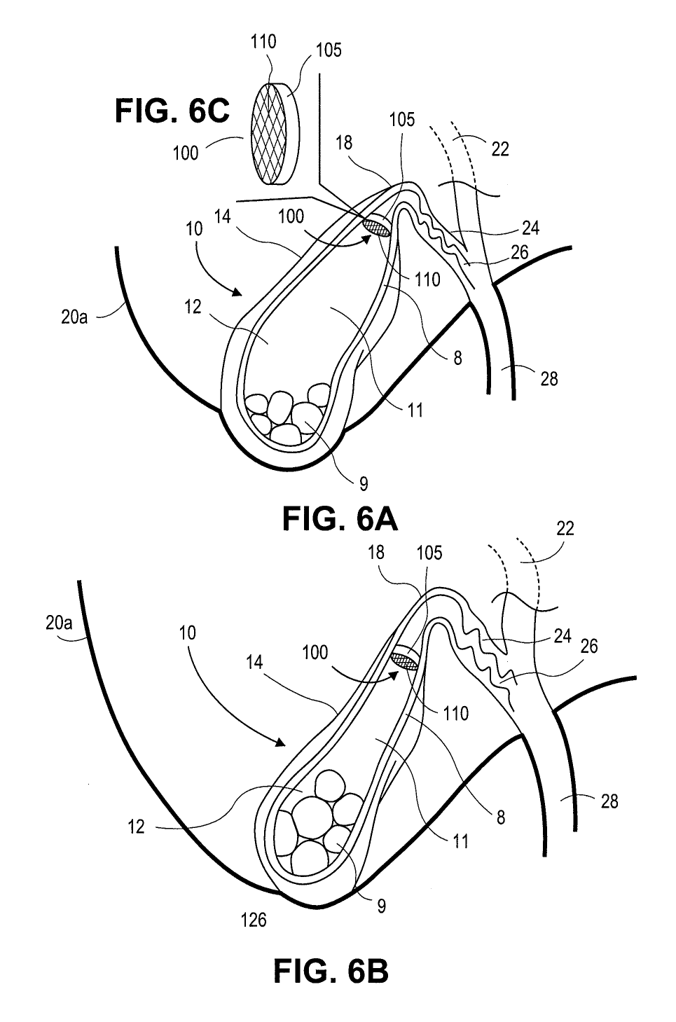

It is to be appreciated that various angles of transverse orientation may be used in the several different aspects of the invention. If measured from a reference of generally orthogonal orientation (i.e., as with dashed lines 12a, 14a) then the angle between the gallbladder central longitudinal axis 19 and gallbladder treatment device may range from 0 degrees in the case of an orthogonal arrangement up to about 50 degrees depending upon the placement of the gallbladder treatment device, the gallbladder anatomy, patient conditions and other factors. Moreover, the gallbladder treatment device maintains an orientation within the gallbladder lumen 11 to prevent gallstones/fragments 9 from entering the neck 18 while the gallbladder is in an expanded condition (see e.g., FIGS. 10A, 10C, 7A, 11A, 11C, and 6A), in a contracted condition (see e.g., FIGS. 10B, 10D, 7B, 11B, 11D, and 6B) and during intermediate conditions between expanded and contracted.

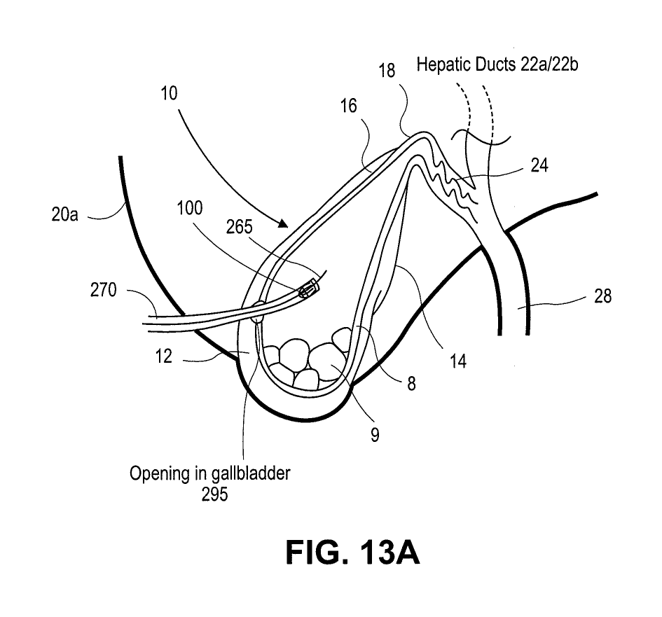

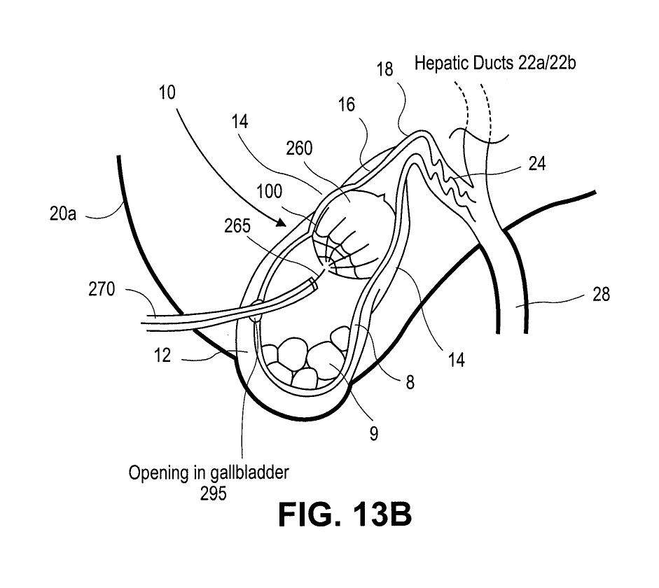

Additional details of the various alternative methods of positioning a gallbladder treatment device within the gallbladder including methods to insert a gallbladder treatment device into the specific sites of intervention listed are also included and described below with regard to FIGS. 13A, 13B, 14, and 15A-15D. In addition, various details of gallbladder anatomy, delivery devices and methods are described in U.S. Patent Application Publication 2009/0143760 entitled “Methods, Devices, Kits and Systems for Defunctionalizing the Gallbladder,” to Jacques Van Dam et al. Turning now to a discussion of gallbladder anatomy and physiology.

### Normal Anatomy and Physiology

The gallbladder 10 is an organ of the digestive tract which stores and concentrates bile by removing water and exchanging electrolytes. The gallbladder functions as a mechanical pump and actively secretes bile through the enterohepatic system to enhance the uptake of lipid-soluble compounds.

### Gallbladder Gross Anatomy

FIG. 1A illustrates the gallbladder 10 within the abdomen and in relation to the liver with a section of the gallbladder wall removed to show the gallbladder interior volume or lumen having gallstones and gallstone fragments 9 therein. The gallbladder 10 is a pear-shaped hollow organ that lies in a depression on the posterior aspect of the right lobe of the liver (segment 5). It measures up to 10 cm long and approximately 3-4 cm wide when fully distended in normal adults. The wall 8 of the gallbladder is 1-2 mm thick. Gallbladder capacity in normal adults is between 30 and 45 ml. The gallbladder 10 is divided into segments: a fundus 12, central body 14, and neck 18, which joins the cystic duct 26. The fundus 12 is the most distal part of the organ and frequently projects beyond the inferior edge of the liver. It is attached to the liver by loose connective tissue and peritoneum covers its free surfaces. The gallbladder is widest at the junction between the fundus 12 and the central body 14. The central body 14 forms the largest segment and tapers to an infundibulum 16 as it joins the neck 18. A peritoneal fold, called the cholecytoduodenal ligament, attaches the exterior of the infundibulum to the first part of the duodenum. The neck is an S-shaped structure, approximately 5-7 mm long, which narrows as it connects with the cystic duct. The various parts of the gallbladder and biliary system are illustrated in FIG. 1A including the spiral valve of Heister 24, the hepatic ducts 22a, 22b, the cystic duct 26 and the common bile duct 28. FIG. 1B is an section of the gallbladder wall 8 showing the relative locations of the various layers with regard to one another and the peritoneum or abdominal wall and the abdominal cavity. The gallbladder wall 8 is made up of an epithelium 1, a lamina propria 2, smooth muscle 3, connective tissue 4, serosa 5. The serosa 5 contacts and is connected to the peritoneum 6 that separates the gall bladder from the abdominal cavity 7.

Gray's Anatomy describes the layers of the gall bladder as having three coats (namely serous, fibrous and muscular) and mucous. The external or serous coat is derived from the peritoneum; it completely invests the fundus, but covers the body and the neck only on their under surface. The fibro-muscular coat is a thin but strong layer which forms the framework of the sac, consisting of dense fibrous tissue which interlaces in all directions and is mixed with plain muscular fibers which are disposed chiefly in a longitudinal direction, a few running transversely. The internal or mucous coat is loosely connected to the fibrous layer. It is generally tinged with a yellowish-brown color, and is everywhere elevated into minute rugae, by the union of which numerous meshes are formed; the depressed intervening spaces having a polygonal outline. The meshes are smaller at the fundus and neck, being most developed about the centre of the sac. (See Gray's Anatomy, A revised American edition from the Fifteenth English Edition, 1977, p. 942).

Although rare, anatomic gallbladder variations can exist, including: congenital absence of the gallbladder +/− cystic duct with a dilated hepatic duct (0.03-0.07% of autopsy cases), irregularly shaped gallbladder including constriction across middle or, rarely, partially longitudinally divided. See Meilstrop, J., Imaging atlas of the normal gallbladder and its variants. 1994: CRC Press. The neck usually follows a gentle curve into the cystic duct but the convexity may be distended into a dilation known as the infundibulum or Hartmann's pouch, usually as the result of chronic obstruction or inflammation. Two distinct gaibladders +/− cystic ducts have been seen in 0.02% of patients. Normally located behind the right lob of the liver along segment 5, the gallbladder is occasionally found to the left of the ligamentum teres or may be intrahepatic, suprahepatic, transversely positioned, floating, beneath the left lobe of the liver or retroperitoneal. Ectopic sites include retrohepatic or in the anterior abdominal wall or falciform ligament. Anomalous pancreatic tissue is sometimes found in the wall of the gallbladder. The fundus may be kinked. A true diverticulum is a rare (0.0008% of all resected gallbladders at the Mayo Clinic) congenital anomaly of the gallbladder and includes all 3 layers of the gallbladder wall. Diverticula of the body and neck may arise from persistent cystohepatic ducts, which run during embryonic life between the gallbladder and liver. The fundal variety arises from incomplete vacuolization of the solid gallbladder during embryonic life. An incomplete septum pinches off a small cavity at the tip of the gallbladder. The congenital variety should be distinguished from pseudodiverticula developing in the diseased gallbladder as a result of partial perforation. Pseudodiverticula in these cases usually contain large gallstones.

Blood supply to the gallbladder is primarily from the cystic artery, which arises from the proximal portion of the right hepatic artery. It branches into superficial channels which lie over the serosa of the gallbladder and deeper channels which travel between the gallbladder and the hepatic bed. Venous drainage consists of small channels on the hepatic side of the gallbladder and other small vessels which merge over the cystic duct and terminate in the portal venous system. Afferent, sympathetic, and parasympathetic nerve fibers from the hepatic plexus supply the gallbladder. Vagal stimulation results in periodic contraction of the smooth muscle of the gallbladder wall. Regional lymph nodes are present at the gallbladder neck and cystic duct and drain to hepatic hilar nodes.

### Gallbladder Histology

The gallbladder wall 8 (from interior to exterior) consists of a surface epithelium, lamina propria, smooth muscle in an overall spiralling pattern along the longitudinal axis of the organ, perimuscular subserosal connective tissue, and serosa. The gallbladder lacks a musclular mucosa and submucosa, which are found in other parts of the digestive tract. The luminal wall is contains primary and secondary folds and the height and width of these folds is variable. The epithelial lining consists of a single layer of columnar epithelial cells above a basement membrane. Epithelial cells have dense microvilli and cilia on their luminal surfaces, with complicated intercellular digitations and infoldings. See Nakanuma, Y., et al., Monolayer and three-dimensional cell culture and living tissue culture of gallbladder epithelium. Microsc Res Tech, 1997. 39(1): p. 71-84. Tubuloalveolar mucous glands are located only in the neck of the gallbladder and consist of cuboid or low columnar cells with abundant clear cytoplasm. The lining cells and mucous glands of the neck contain sulfated acid mucin.

### Cystic Duct Anatomy

The cystic duct 26 joins the common hepatic duct roughly 2 cm distal to the confluence of the right and left hepatic ducts. The mean length of the cystic duct in humans is 30 mm and the mean collapsed diameter is 4 mm. See Frierson, H. F., Jr., The gross anatomy and histology of the gallbladder, extrahepatic bile ducts, Vaterian system, and minor papilla. Am J Surg Pathol, 1989. 13(2): p. 146-62. The cells lining the cystic duct are morphologically identical to those inside the gallbladder. Where the cystic duct joins the gallbladder neck, there are large, oblique folds which contain bundles of smooth muscle believed to prevent overdistension and collapse of the cystic duct during changes in pressure. This area of smooth muscle folds is referred to as the spiral valve of Heister.

### Normal Gallbladder Physiology

The gallbladder excretes 500-1000 cc of bile per day. Bile is concentrated 5-10× in humans by the active absorption of electrolytes accompanied by the passive movement of water. Bile is critical for the intestinal absorption of dietary fat and consists of bile salts, lecithin, cholesterol and organic solutes. Bile salts are the major products of cholesterol metabolism and part of a family of water-soluble sterols which are highly effective detergents capable of solubilizing lipids secreted by the liver (usually lecithin) into the biliary tree to promote lipid absorption in the gut. Lecithin is a hydrophobic, non-aqueous compound with minimal solubility in water. Cholecyetikinin, a hormone secreted by intestinal cells, causes contraction of the gallbladder and relaxation of the sphincter of Oddi in response to a meal.

The fundus 12 and body 14 of the gallbladder are the widest and most contractile parts of the organ. Stones 9 form and reside predominately within the lower portion of the fundus 12. FIG. 1A illustrates the gallbladder within the abdomen and in relation to the liver with a section of the gallbladder wall removed to show the gallbladder interior volume or lumen having gallstones and gallstone fragments therein. FIG. 1 illustrates the various parts of the gallbladder with stones pooled in the fundus 12. In one embodiment, a gallbladder treatment device may be placed in the fundus or body to prevent passage of gallstones into the neck or infundibulum.

### Gallbladder Treatment Device

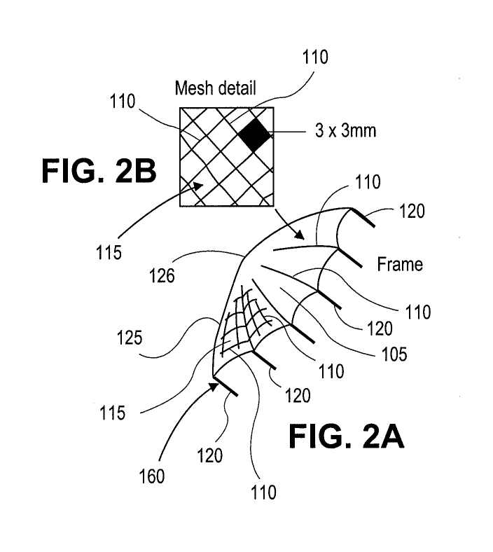

FIGS. 2A and 2B are isometric and enlarged views, respectively, of an embodiment of a gallbladder treatment device. The filter is composed of a filtering mesh and a framework of struts which hold the filter in place against the gallbladder wall. It is known clinically that stones of <3 mm will pass easily through the elastic 1-5 mm wide cystic duct without obstructing. Therefore the filtering mesh is porous to allow passage of any particles <3 mm in diameter. The filter is flexible and changes conformation when the gallbladder contracts. The body and fundus are the most contractile portions of the gallbladder. The filter must undergo a significant conformational change in order to collapse to the diameter of the fully contracted organ. FIGS. 6B, 7B, 10B, 10D, 11B, and 11D shows various gallbladder treatment device embodiments within a contracted gallbladder 10. The porosity or filtering characteristic of the element or elements 110 arranged to prevent gallstone 9 passage (i.e., in one configuration as a filtering mesh) does not change when the gallbladder treatment device collapses with the contracting gallbladder 10. As the diameter of the gallbladder body decreases with contraction, the struts 120 of the gallbladder treatment device come closer together and the filtering mesh collapses distally into or towards the gallbladder fundus. The gallbladder treatment device decreases in diameter and increases in profile length during contraction.

The plurality of elements 110 to prevent gallstone passage may be configured as a plurality of intersecting elements forming a filter, or a filtering mesh or other arrangement attached to a frame 105. The frame 105 may be a flexible or rigid frame, for example of nitinol or stainless steel.

FIG. 2B is an enlarged view of the gallbladder treatment device 100 of FIG. 2A. FIG. 2B illustrates the relationship between a frame 105 formed from at least one element 110 having a filtering characteristic 115 dimensioned to prevent the passage of a gallstone or gallstone fragment 9 having a largest dimension of greater than 3 mm. In one specific example, plurality of elements with a filtering characteristic to prevent passage of gallstones or gallstone fragments greater than 3 mm is a porous filtering mesh attached to a frame 105 and struts formed from nitinol. The mesh is connected to the frame and forms a cross-sectional filtering surface. The porosity of the mesh is <3 mm. In one specific embodiment, the filter has a pore size or filter capacity sized to prevent passage of gallstones or gallstone fragments less than about 3 mm in the largest dimension. In another specific embodiment, the filter has a pore size or filter capacity sized to prevent passage of gallstones or gallstone fragments less than about 2 mm in the largest dimension. In another specific embodiment, the filter has a pore size or filter capacity sized to prevent passage of gallstones or gallstone fragments less than about 1 mm in the largest dimension.

In one embodiment, there is a gallbladder treatment device 100 for maintaining a gallstone or a gallstone fragment 9 within a portion of a gallbladder distal to a gallbladder neck. A frame 105 is formed from at least one element 110 having a filtering characteristic 115 dimensioned to prevent the passage of a gallstone or gallstone fragment having a largest dimension of greater than 3 mm. There are a plurality of struts 120 extending from the frame 105 to secure the frame 105 in an orientation transverse to a gallbladder central, longitudinal axis 19. As such the gallstones or the gallstone fragments 9 having a largest dimension of greater than 3 mm will remain within the gallbladder 10 on one side of the frame 105. In one aspect, the frame 105 has a generally rounded contour 126 with a radius of curvature or a frame contour 125 between about 0.1 cm to about 1.0 cm. In still another aspect, the frame has a generally conical contour 127 with a radius of curvature or a frame contour 125 between about 0.1 cm to about 1.0 cm. In still other alternative embodiments, the at least one element is a flexible sheet of material and the filtering characteristic is a plurality of apertures formed in the flexible sheet having a largest dimension of less than 3 mm. In still other embodiments, the at least one element is a plurality of flexible strands 136 from a braided structure 135 that maintains a filtering characteristic that is the spacing between adjacent strands that is less than 3 mm. In still another alternative, the at least one element is a plurality of suture strands 140 attached to the frame to form a filter structure that maintains a filtering characteristic that is the spacing between adjacent suture strands that is less than 3 mm.

A variety of different filter structures may be employed to retain material greater than 3 mm within the fundus thereby reducing the risk of impaction. The filtering component may be braided nitinol, a thin sheet of flexible material such as nitinol or a porous inorganic material such as ePTFE. The thin sheet may be treated to formed with pores or apertures are stamped or punched out. In still another aspect, a filter structure is a suture-based net. The frame, struts, filters, filtering elements or filtering material can be rigid metal such as nitinol, or thin and flexible if constructed from nitinol sheet, braid, or a suture-based net. The pores, apertures or openings formed on or in the device can change size with contraction of the device. For example a nitinol braid filter would have increased pore size when the organ expands and decreased pore size as the organ contracts. Since stones and fragments are most likely to obstruct during organ contraction, pore size can shrink during contraction to prevent stones from passing and increase during expansion to maximize bile filling and flow.

In still another aspect, there is another gallbladder treatment device for maintaining a gallstone or a gallstone fragment within a portion of a gallbladder distal to a gallbladder neck. This device has a plurality of struts having a first end and a second end. There is a curved portion between the first end and the second end moveable between a first condition having a radius of curvature induced by a contracted gallbladder and a second condition having a radius of curvature induced by an expanded gallbladder. The plurality of struts overlap one another at least once between the first end and the second end such that the resulting pattern of overlapping struts prevents the passage of a gallstone or gallstone fragment greater than 3 mm.

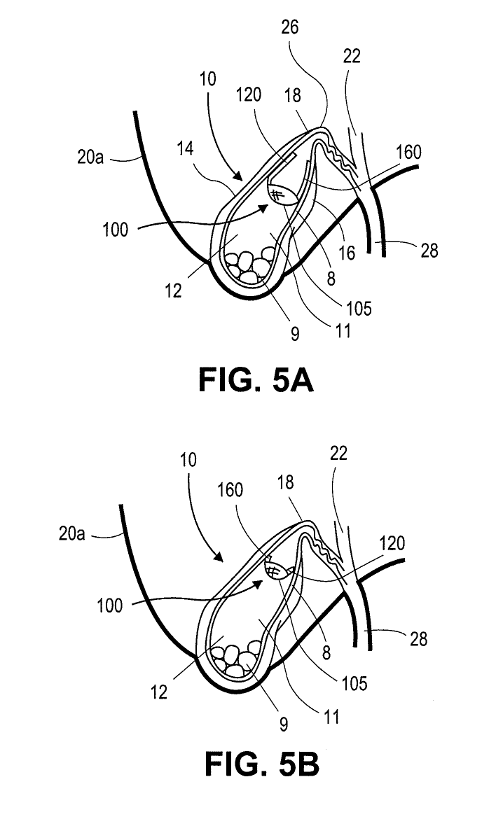

The rigid frame can be designed to flex at certain points, for example at the point of contact with the gallbladder wall or in the center of the lumen, in order to collapse and contract with contraction of the organ. The frame may be held in place inside the gallbladder organ using radial force or a fixation technique. The amount of radial force required to hold the filter in place may be decreased by altering the shape and wall-contacting surface of the struts to improve adhesion and cell in-growth. As shown for example in FIGS. 5A and 5B, strut length may be adjusted to increase or decrease the surface area of the strut in contact with the luminal wall or strut wall contact portion. An increase in contacting surface area may aid in positioning and anchoring the device during active contraction. Adding materials or surface finished to the wall-contacting parts of the struts may also improve adhesion and anchoring. For example, coating the wall-contacting surface of the strut with a biologic material known to increase fibrous tissue in-growth, such as PLGA, could improve adhesion and anchoring. Other suitable materials for promoting growth are polyethyleneglycol and epithelial growth factor. Coating the contacting surface with a porous inorganic material such as ePTFE may also improve adhesion and anchoring. Surface modifications without adding a new material may also aid in adhesion, for example adding small spikes or grooves to the wall-contacting strut surface.

Alternatively or in addition to radial forces, a device can maintain position within the gallbladder using one or a variety of mechanisms. Various different mechanisms and techniques will be described with regard to FIGS. 3A-3D.

FIG. 3 illustrates the gallbladder as shown in FIG. 1A with a section of the gallbladder wall removed to show an exemplary gallbladder treatment device within the gallbladder interior. The device is positioned in an orientation to maintain the gallstones or gallstone fragments on one side of the device and away from the gallbladder neck.

FIG. 3A is an enlarged section view of a strut 120 of the device in FIG. 3. This view shows a wall contacting section 160 of the strut 120 along a portion of the gallbladder wall 8.

Struts 120 composing the frame of the device as shown in FIG. 3A may use radial force alone, which do not penetrate any layers of the gallbladder wall. Because purchase in the wall is limited, struts of this type will only be fully anchored once tissue in-growth occurs, which can take days to weeks to mature. Additionally or alternatively, the strut or wall contacting portion may also include a fixation element 142 such as those in FIGS. 3B, 3C and 3D.

FIG. 3B is an enlarged view of a strut 120 of the device in FIG. 3 showing a wall contacting section 160 of the strut 120 with fixation elements 142. Here the fixation elements are used within the gallbladder along a portion of the gallbladder wall. In this embodiment, the fixation elements such the short spikes or barbs 144 that penetrate at least partially into the gallbladder wall 8.

FIG. 3B illustrates self-inserting partial thickness barbs or tacks 144, which will have limited purchase in the gallbladder wall 8 but enough to reduce the amount of radial force necessary to hold the device in place until tissue in-growth is complete. The benefit of partial thickness anchoring is there is no risk of perforation or leak/contamination of the peritoneum.

FIG. 3C is an enlarged section view of a strut of the device in FIG. 3 showing a wall contacting section 160 of the strut 120 with fixation elements 142 used for through the gallbladder wall fixation along a portion of the gallbladder wall. In this embodiment, such that the clips 144 penetrate completely through the gallbladder wall 9.

FIG. 3C illustrates full-thickness barbs or tacks 144 which can hold the device 100 in place without or with little radial force or tissue in-growth needed. The wall puncturing is small, for example less than 0.01″ in diameter, and if the fixation element is not removed from the wall, the risk leaking/contamination is limited.

FIG. 3D is an enlarged section view of a strut 120 of the device in FIG. 3 showing a wall contacting section 160 of the strut 120 having an opening 148. The opening 148 permits a separate fixation element 142 to pass though the opening 148 and then partially or completely through the gallbladder wall 8. One or more openings 148 may be formed in a portion of a wall contacting section 160 to permit passage of one or more fastening devices or one or more elements of a single fixation device.

FIG. 3D illustrates separately applied barbs, suture, or tacks. This anchoring method would have risks and benefits similar to the full-thickness barbs described in FIG. 3C.

In one gallbladder treatment device alternative, a portion of each strut 120 has an opening 148 to permit passage of a fixation element 142. The opening may be in the wall contacting section. The fixation element 142 is sized to pass through the opening 148 and has a length to at least partially penetrate a gallbladder wall 8. In another aspect, the fixation element has a length sufficient to penetrate completely through a gallbladder wall.

If radial force is employed, it must be less than the contractile force exerted by the gallbladder wall to reduce or prevent penetration or erosion through the wall 8. Multiple points of contact with the gallbladder wall may be used to distribute radial forces at rest and during contraction and expansion of the organ. In one gallbladder treatment device alternative, the plurality of struts 120 are shaped to deflect outwardly against a wall 8 with a radial force sufficient to maintain the frame in the orientation transverse to a gallbladder central, longitudinal axis 19. Moreover, the gallbladder treatment device maintains a transverse orientation when the gallbladder is in a contracted condition and an expanded condition.

The thickness of the gallbladder wall is approximately 1 mm. If partial thickness tacks or barbs are employed, they must penetrate less than 0.75 mm or ¾ of the total wall thickness to limit the risk of full-thickness puncture. If full-thickness barbs or tacks are employed, patches of Dacron, ePTFE or a similar material may be inserted between the struts of the device and the gallbladder wall to prevent leaking through the puncture site into the abdominal cavity. Additional struts or rings may extend from the device along the walls of the fundus or body of the gallbladder. These extensions may act to distribute forces generated during contraction or provide anchoring assistance.

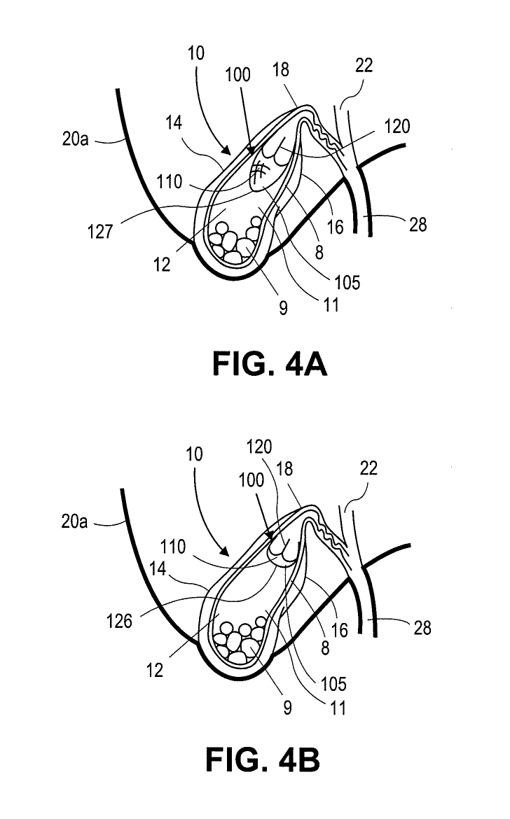

FIG. 4A illustrates the gallbladder as shown in FIG. 1A with a section of the gallbladder wall 8 removed to show an exemplary gallbladder treatment device. This device has a conical 127 frame contour 125. The device is within the gallbladder interior positioned in an orientation to maintain the gallstones or gallstone fragments on one side of the device and away from the gallbladder neck.

FIG. 4B illustrates the gallbladder as shown in FIG. 1A with a section of the gallbladder wall 8 removed to show an exemplary gallbladder treatment device 100. This device has a rounded 126 frame contour 125 within the gallbladder interior 11 positioned in an orientation to maintain the gallstones or gallstone fragments 9 on one side of the device 100 and away from the gallbladder neck 18.

FIG. 5A illustrates the gallbladder 10 as shown in FIG. 1A with a section of the gallbladder wall 8 removed to show an exemplary gallbladder treatment device 100. This device has a rounded 126 frame contour 125 and struts 120 adjusted to increase the wall contacting section 160 within the gallbladder interior positioned in an orientation to maintain the gallstones or gallstone fragments on one side of the device and away from the gallbladder neck;

FIG. 5B illustrates the gallbladder as shown in FIG. 1A with a section of the gallbladder wall removed. In this exemplary gallbladder treatment device 100 there is a rounded frame contour and struts adjusted to decrease the wall contacting section 160 within the gallbladder interior 11. The device is positioned in an orientation to maintain the gallstones or gallstone fragments 9 on one side of the device 100 and away from the gallbladder neck 18.

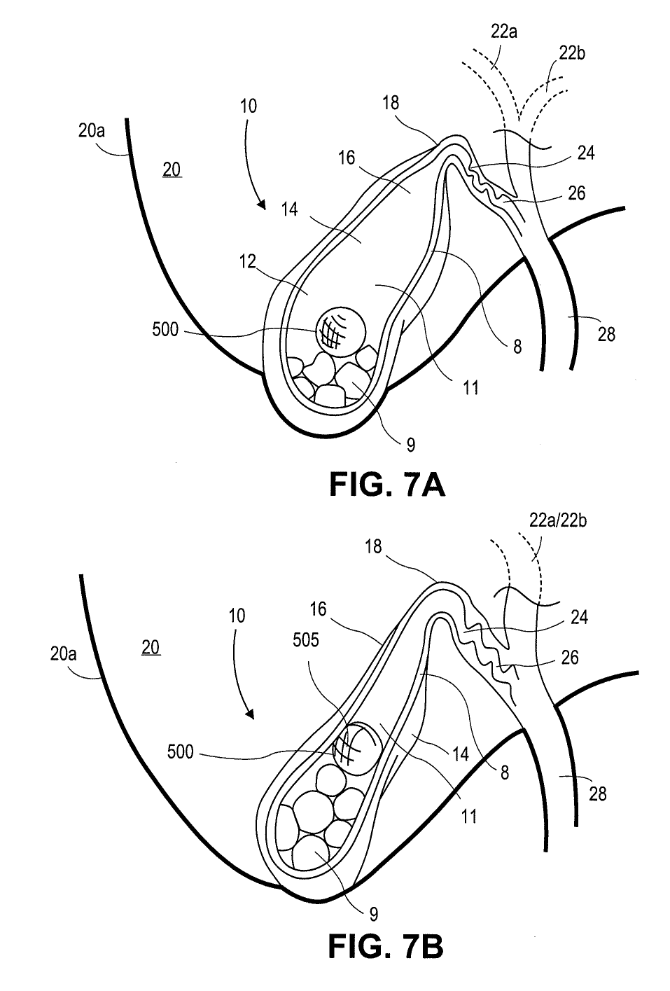

In one aspect, there are gallbladder treatment devices for maintaining a gallstone or a gallstone fragment within a portion of a gallbladder distal to a gallbladder neck having a plurality of struts 120 arranged in a generally cylindrical pattern. A wall contacting section 160 is provided on each of the plurality of struts. A frame 105 formed from a plurality of flexible members 110 attached between two of the plurality of struts 120. The spacing between two adjacent flexible members 110 when attached between two of the plurality of struts 120 is less than 3 mm. When in use in the gallbladder, the frame 105 maintains the gallstone or the gallstone fragments 9 within a portion of a gallbladder lumen 11 distal to a gallbladder neck 18. Moreover, the radius of curvature 125 formed by the plurality of flexible members 110 is variable between a first curvature when the plurality of struts 120 are contacting an expanded gallbladder and a second curvature when the plurality of struts 120 are contacting a contracted gallbladder. Examples of first curvatures may be found in, for example, FIGS. 10A, 10C, 7A, 11A, 11C, 5A, and 5B. Examples of second curvatures may be found in, for example, FIGS. 10B, 10D, 7B, 11B, and 11D.

FIGS. 6A and 6B illustrate the gallbladder as shown in FIG. 1A in an expanded and contracted condition, respectively, with a section of the gallbladder wall removed to show an exemplary hoop framed gallbladder treatment device 100. the device 100 (best seen in FIG. 6C) is within the gallbladder infundibular interior and positioned in an orientation to maintain the gallstones or gallstone fragments 9 on one side of the device 100 and away from the gallbladder neck 18.

In this embodiment, the gallbladder treatment device 100 has a hoop or ring shaped frame 105 to support at least one element 110 with a filtering characteristic dimensioned to maintain a gallstone or gallstone fragment on one side of the device. In the illustrated embodiment of FIG. 6C, the at least one element 110 is a plurality of strands arranged in a regular pattern to maintain a filtering characteristic of with spacing of 3 mm or less between adjacent strands 110. The frame 105 is illustrated as a hoop or short cylinder where the diameter of the hoop is the same on both edges. The height of the wall of the frame may vary or be sized according to the implant site within the gallbladder. In addition, the walls need not be flat as shown but may also be angled or tapered. In this case, one side of the hoop would have a first diameter and the other side would have a second diameter and the first and second diameters would be different. Using different diameters and a gradual slope between them will produce a tapered frame 105. Other configurations are possible such as intermediate diameters between the first side and the second side to produce an hourglass shape frame 105.

FIGS. 6A and 6B illustrate the gallbladder treatment device 100 with a circumferential ring frame positioned within the gallbladder infundibular during gallbladder expansion (FIG. 6A) and contraction (FIG. 6B). Because the infundibular diameter does not change significantly but instead remains with the normal size range of from 1.5-2 cm during contraction, the hoop frame design does not undergo a conformational change when the gallbladder contracts, as depicted in FIG. 6B.

The infundibulum tapers as it approaches the neck and outlet. Tapering the outer walls of the frame as discussed above may be used to produce a variety of different dimensioned tapered wall gallbladder treatment devices. It is believed that tapered hoop walls to substantially conform the hoop to the contours, shape or taper of the gallbladder infundibulum will improve positioning.

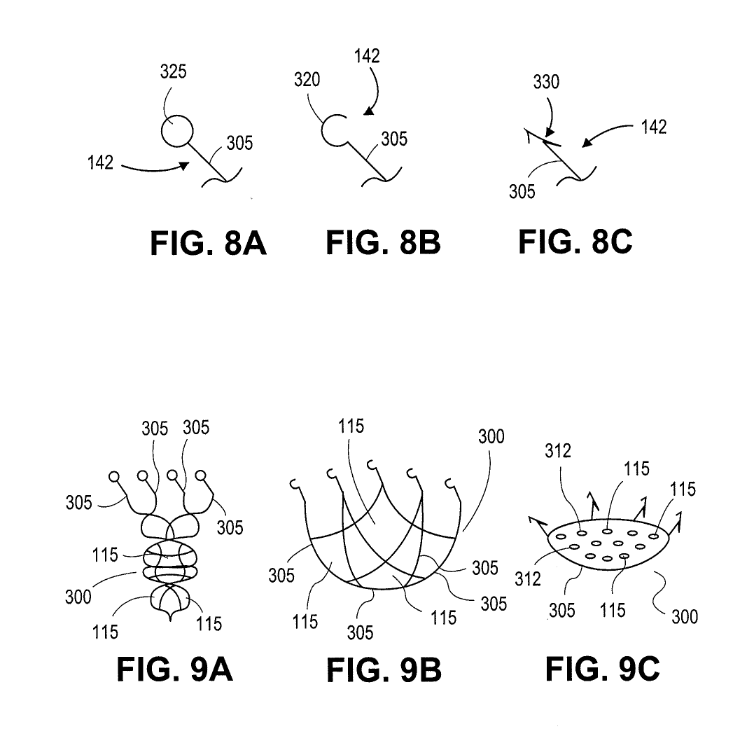

As with other devices described herein, the hoop frame may remain in position by one or more of the use of radial force, over sizing hoop frame, or tapering the frame sidewalls. Moreover, self-inserting barbs or tacks, or separately applied barbs or tacks along the hoop walls may be used to anchor the hoop to the infundibular wall. The hoop may also be attached to the gallbladder wall 8 using one or more strands of suture and conventional suturing techniques. The materials and construction techniques described above as well as the fixation techniques described herein in relation to FIGS. 3, 3A, 3B, 3C, 3D, 8A, 8B and 8C may also be used in the construction of a gallbladder treatment device configured, sized and adapted for placement in the gallbladder infundibulum.

FIGS. 7A and 7B illustrate the gallbladder as shown in FIG. 1A in an expanded and contracted condition, respectively, with a section of the gallbladder wall removed to show an exemplary frameless gallbladder treatment device 500 (shown here a mesh sphere). The device 500 is within the gallbladder fundus interior positioned in an orientation to maintain the gallstones or gallstone fragments 9 on one side of the device 500 and away from the gallbladder neck.

Another example of a gallbladder treatment device which can be placed in the body of the gallbladder is free-floating in or on top of bile. This design is not anchored to the walls of the gallbladder. FIG. 7A illustrates a sphere-shaped filter 500 floating in bile in the fundus of the gallbladder at rest. During contraction, as described in FIG. 7B, the filtering sphere 500 is trapped as the gallbladder narrows. Fluid and particles <3 mm can pass through the filtering sphere. Particles >3 mm remain distal to the filtering sphere in the gallbladder fundus.

FIGS. 7A and 7B illustrate an embodiment of a gallbladder treatment device 500. In the device 500, the plurality of struts 120 are shaped to conform to a portion of the curvature of a wall of the gallbladder fundus. The conformal strut shape maintains the frame in a portion of the gallbladder fundus in an orientation transverse to a gallbladder central, longitudinal axis 19 in order to maintain the gallstones or the gallstone fragments 9 having a largest dimension of greater than 3 mm on one side of the frame and within a more distal portion of the gallbladder fundus. This embodiment also represents a gallbladder device alternative where the plurality of struts 120 are shaped into a closed or substantially closed three dimensional structure. In one aspect, the closed or substantially closed three dimensional structure corresponds to one or more curvatures within the gallbladder lumen 11. In another aspect, the closed or substantially closed three dimensional structure may have an expanded shape or configuration in an expanded gallbladder and convert to a partially or fully deformed shape under the forces applied by a contracting gallbladder (see FIG. 7B).

In the illustrated embodiment, the expanded shape is generally spherical as shown in FIG. 7A. The deformed shape in FIG. 7B shows how the gallbladder walls have pressed in the sides of the sphere giving it a more oblong or ovoid shape. It is to be appreciated that other shapes are possible for these embodiments. In one aspect, the closed or substantially closed three dimensional structure is generally shaped as a sphere. In still another aspect, the closed or substantially closed three dimensional structure or an ovoid that is dimensioned to fit within the gallbladder interior 11.

FIG. 8A illustrates an exemplary gallbladder treatment device 300 formed from at least one element 305 having a filtering characteristic 115 dimensioned to prevent the passage of a gallstone or gallstone fragment 9 having a largest dimension of greater than 3 mm. The device 300 shown here is a braided structure 135 with loop 325 shaped fixation elements 142 (FIG. 9A) spaced along and attached to the perimeter of the braided structure 135.

FIG. 8B illustrates an exemplary gallbladder treatment device structure 300 formed from at least one element 305 having a filtering characteristic 115 dimensioned to prevent the passage of a gallstone or gallstone fragment 9 having a largest dimension of greater than 3 mm. The device 300 shown here is a mesh filter of intersecting strands with hook fixation elements 320 (seen best in FIG. 9B). The hooks 320 are spaced along and attached to the perimeter of the filter elements 305. The intersecting strands 305 may be formed from a suitable suture material.

FIG. 8C illustrates an exemplary gallbladder treatment device structure 300 formed from at least one element 305 having a filtering characteristic 115 dimensioned to prevent the passage of a gallstone or gallstone fragment 9 having a largest dimension of greater than 3 mm. The device 300 shown here is a perforated flexible sheet having openings 312 formed therein. The filtering characteristic 115 is provided by selecting the shape, size, number and spacing of the perforations or openings 312. This device is shown with pivoting barbed tip fixation elements 330 (shown best in FIG. 9C). The elements 330 are spaced along and attached to the perimeter of the flexible sheet.

FIG. 9A illustrates a loop shaped fixation element 325 as shown with the gallbladder treatment device of FIG. 8A.

FIG. 9B illustrates a hook shaped fixation element 320 as shown with the gallbladder treatment device of FIG. 8B.

FIG. 9C illustrates a pivoting barbed tip fixation element 330 as shown with the gallbladder treatment device of FIG. 8C. The barb element is attached in a pivoting or flexible manner to the at least one element 305. In this way, the barb may be in line for penetration and then tee across the element 305 to hold onto the wall.

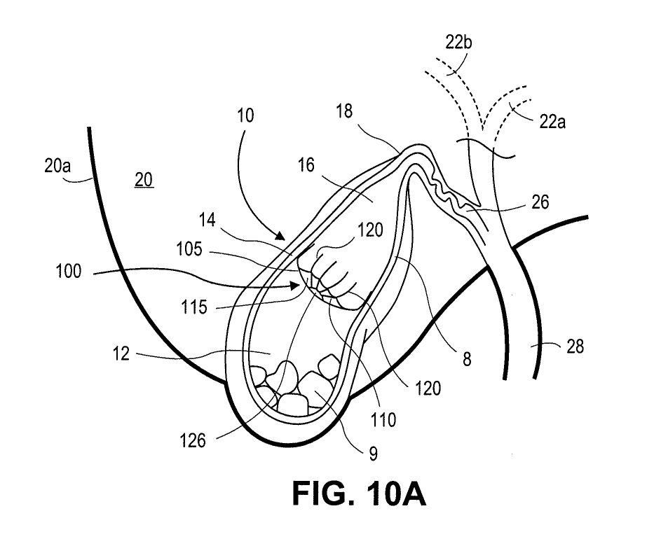

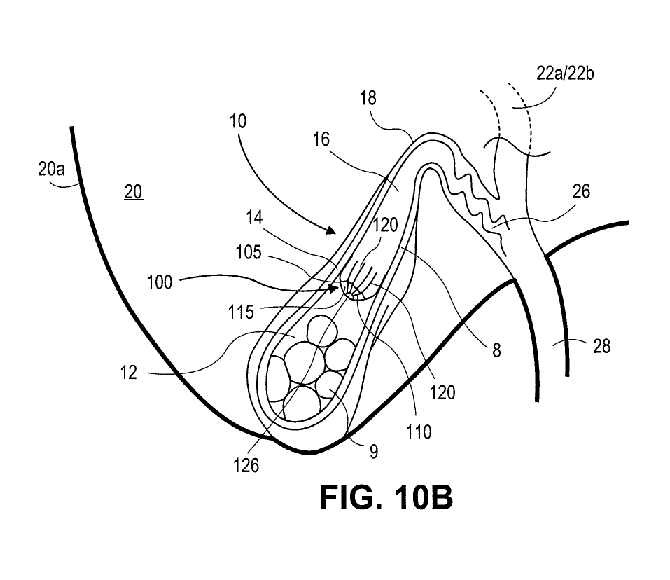

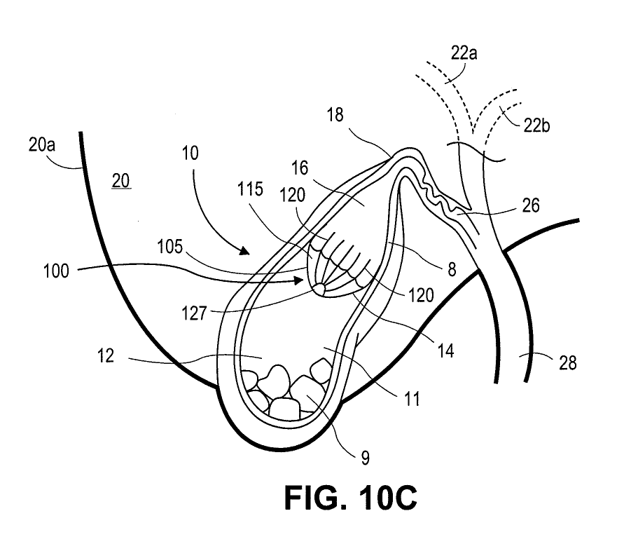

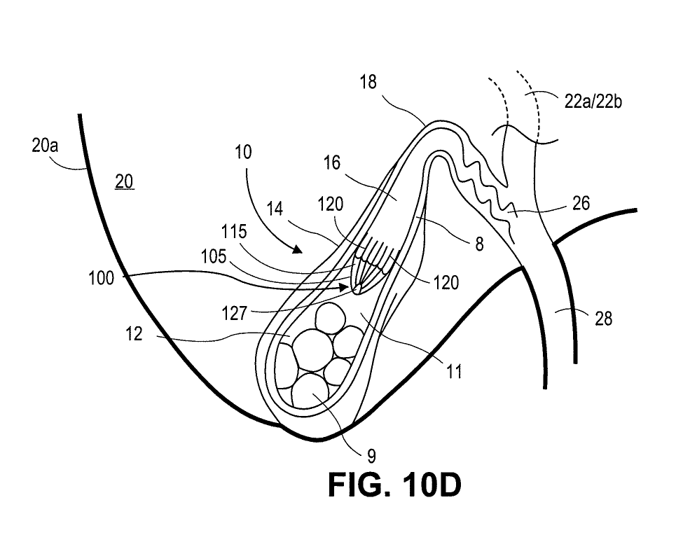

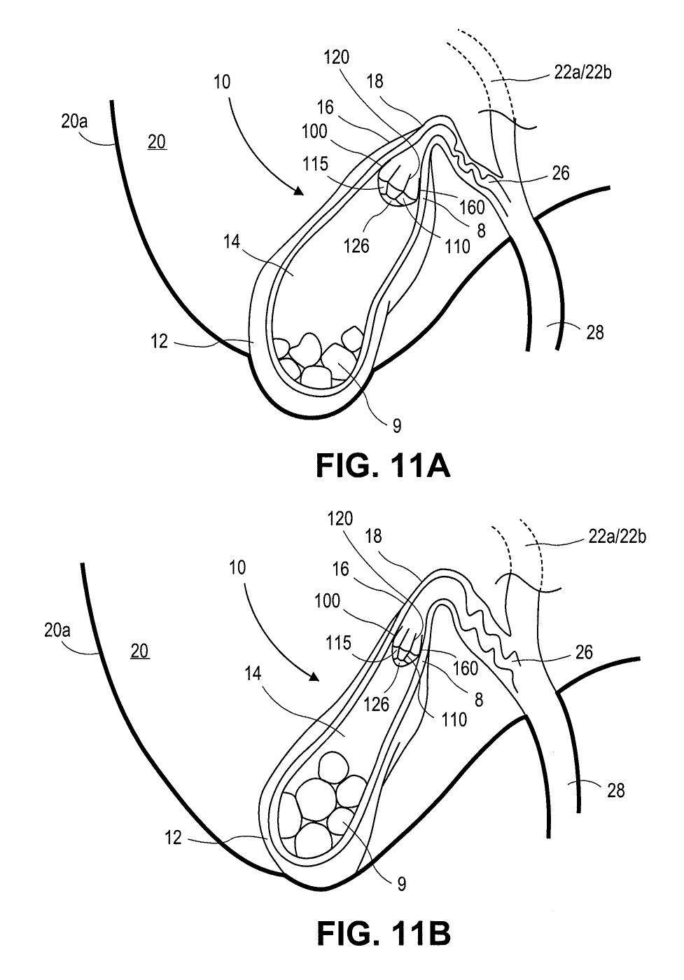

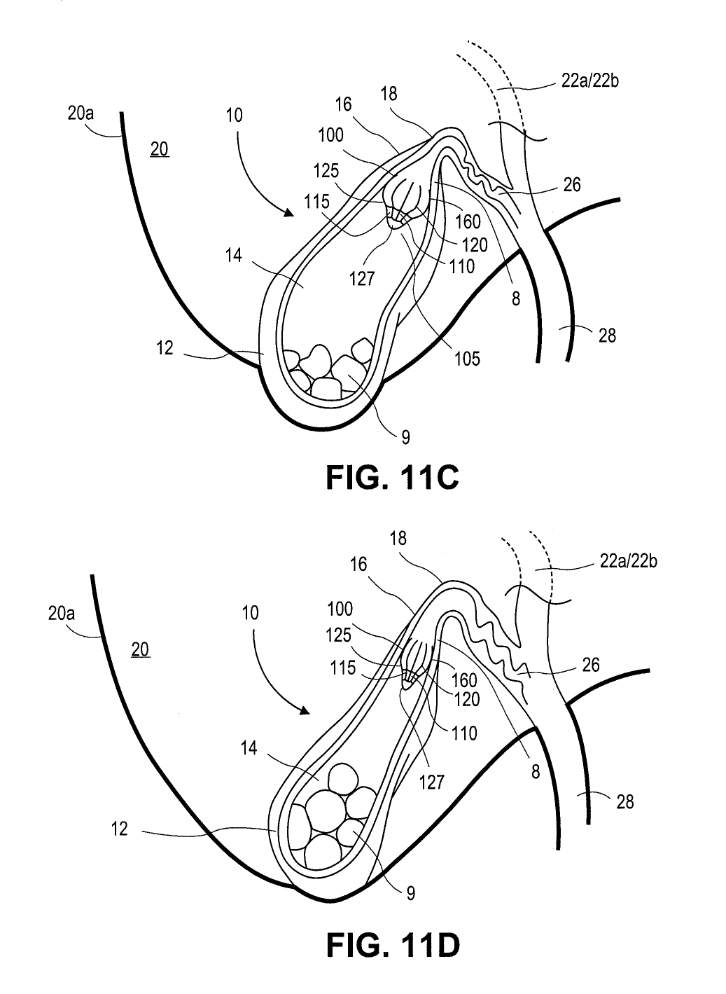

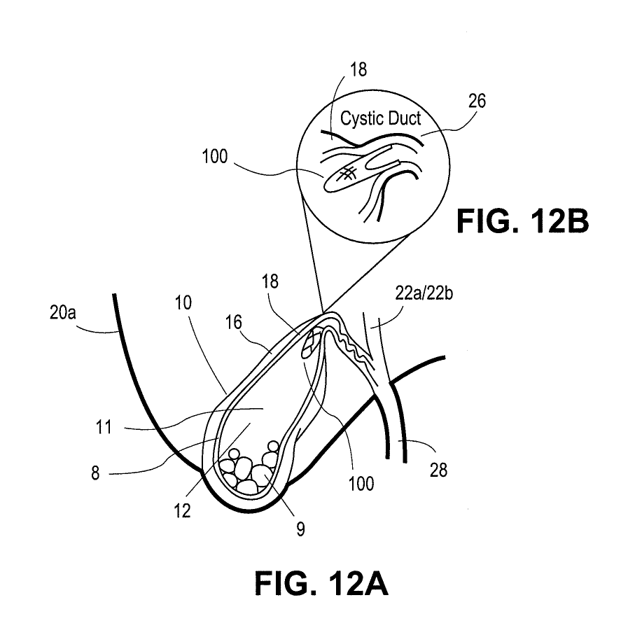

It is to be appreciated that the alternative gallbladder treatment devices 300 of FIGS. 8A, 8B and 8C (as with other gallbladder treatment devices 100, 500 described herein) are structures dimensioned to span a portion of the gallbladder transverse to the gallbladder central longitudinal axis. In one aspect, the portion of the gallbladder comprises a portion of the gallbladder fundus 12. In another aspect, the portion of the gallbladder comprises a portion of the gallbladder body 14 (see for example FIGS. 10A-10D). In still other embodiments, the portion of the gallbladder comprises a portion of the gallbladder infundibulum 16 (see for example FIGS. 11A-11D). In still other embodiments, the portion of the gallbladder comprises a portion of the gallbladder neck 18 (see for example FIGS. 12A and 12B).

Still further, it is to be appreciated that the alternative gallbladder treatment devices 300 of FIGS. 8A, 8B and 8C (as with other gallbladder treatment devices herein) also illustrate gallbladder treatment devices having one or more fixation elements spaced about a perimeter of the device. In these specific embodiments, the various alternative fixation devices are generally evenly spaced around the perimeter. Moreover, the perimeter is along a generally distal edge. Other locations are possible such as along a sidewall or set back from an edge depending on a number of factors such as the specific fixation device being used, the desired device gallbladder implant location and other factors. The one or more fixation elements may include a hook 320 as shown in FIGS. 8B and 9B. The one or more fixation elements may include a ring 325 as shown in 8A and 9A. The one or more fixation elements may include a pivoting barbed tip 330 as shown in FIGS. 9C and 8C. Still further, the one or more fixation elements may additionally or alternatively include a strand of suture. Moreover, in additional or alternative embodiments, the fixation elements and techniques described herein may be used alone or in any combination as needed for the particular gallbladder treatment device and gallbladder implant location, for example, as well as other factors.

### Materials for Use in the Gallbladder

A device or material placed inside the gallbladder will be subject to biliary and biofilm deposition as well as cellular overgrowth at points of contact with the luminal wall of the gallbladder. Bile tolerance and biofilm resistance are the most important characteristics to maintain gallbladder treatment device patency over long-term implantation within the gallbladder.

Bile is acidic and may contain free-floating cholesterol crystals and calcium or bilirubinate particles. Biliary deposits are primarily the result of cholesterol crystal precipitation, often in conjunction with or following biofilm, glycoprotein, or calcium bilirubinate deposition. To resist biliary and cholesterol deposition, hydrophillicity and low porosity are the most important characteristics. Nitinol and stainless steel have been shown to resist biliary deposition. Electropolishing these materials will remove oxide and further decrease deposition risk by reducing surface porosity. Electropolishing also reduces the risk of stress fracture at points of flexion in the device. Decreasing porosity and limiting the number of sharp or penetrating edges and barbs may also aid in removal of the device after implantation. However, increased porosity at the point(s) of contact between the device and the gallbladder wall may aid in cellular overgrowth and strengthen the anchoring force of the device. In other aspects of a gallbladder treatment device embodiment, at least a portion of the frame or a portion of the struts is formed from materials having substantially non-porous, hydrophillic surfaces.

Biofilm formation is expected to occur on virtually all implants located within the gastrointestinal tract, regardless of material and geometry. Biofilm formation can be delayed and in some cases prevented by surface modifications or material choices. This includes the use of antibiotic coated or antibiotic eluting materials, hydrophillic coatings, and low-porosity surfaces. Examples of materials which resist biofilm include FEP, ePTFE, and hydromer coating which are already used in biliary stents such as those available from W. L. Gore and Bard Medical. Stainless steel, elgiloy, and nitinol are naturally resistant to some biofilms. In some other aspects of the gallbladder treatment device, at least a portion of the frame or a portion of the struts is coated with a material to inhibit gallbladder biofilm formation. In one alternative, the material is an antibiotic selected from the group of antibiotics suited to treat infections of the gastrointestinal tract. In one specific example, the antibiotic is a ciprofloxacin.

Adding sharp or protruding edges to the parts of the device which come in contact with free floating gallstones may promote breakdown or these stones through mechanical abrasion. Coating the device with drug or a bile acid, for example chenodeoxycholic or ursodeoxycholic acid may aid in chemical dissolution of existing gallstones and prevent the formation of future stones.

In some variations, the fixation element is formed in or attached to a portion of the strut each having a surface treatment 146 to encourage fibrous in-growth. The surface treatment is a coating of a growth promoting compound, in some embodiments. The growth promoting compound may include polyethyleneglycol. Additionally or alternatively the growth promoting compound includes an epithelial growth factor.

### Gallbladder Treatment Device Alternatives Corresponding to Anatomic Location

FIGS. 10A and 10B illustrate the gallbladder 10 as shown in FIG. 1A in an expanded and contracted condition, respectively, with a section of the gallbladder wall removed to show an exemplary gallbladder treatment device 100. The device 100 has a rounded 126 frame contour 125 within the gallbladder body 14 interior. The device 100 is positioned in an orientation to maintain the gallstones or gallstone fragments 9 on one side of the device 100 and away from the gallbladder neck 18.

FIG. 10A depicts a device 100 placed in the body 14 of the gallbladder. The filter is composed of a filtering mesh 110 and a framework of struts 120 which hold the filter (formed from elements 110) in place against the gallbladder wall 8. It is known clinically that stones of <3 mm will pass easily through the elastic 1-5 mm wide cystic duct without obstructing. Therefore the filtering mesh is porous to allow passage of any particles <3 mm in diameter. The device 100 is flexible and changes conformation when the gallbladder contracts. The body 14 and fundus 12 are the most contractile portions of the gallbladder 10. The device 100 must undergo a significant conformational change in order to collapse to the diameter of the fully contracted organ.

FIG. 10B shows the device 100 in a collapsed configuration inside a contracted gallbladder 10. The porosity of the filtering mesh does not change when the filter collapses. As the diameter of the gallbladder body 14 decreases with contraction, the struts 120 of the device 100 come closer together and the filtering mesh collapses distally into the gallbladder fundus 12. The device 100 decreases in diameter and increases in profile length during contraction.

FIGS. 10C and 10D illustrate the gallbladder as shown in FIG. 1A in an expanded and contracted condition, respectively, with a section of the gallbladder wall removed to show another exemplary gallbladder treatment device 100. In this embodiment, the device 100 has a conical 127 frame contour 125 within the gallbladder body 14 interior positioned in an orientation to maintain the gallstones or gallstone fragments 9 on one side of the device 100 and away from the gallbladder neck 18.

The infundibulum 16 is a narrow, tapered area between the gallbladder body 14 and neck 18. It is typically 1-3 cm wide at its origin and contains less smooth muscle than the fundus and body. The cross-sectional area of the infundibulum 16 changes less during contraction than the fundus or body, which can change both shape and size, decreasing in volume up to 70% with contraction. A device positioned in the neck or infundibulum will be affected less by gallbladder contractions. The cross-sectional area of the infundibulum also varies less across people than the body or fundus of the gallbladder. Gallbladder overall length and width can vary up to 2-3 cm between adult patients, while neck, infundibulum, and cystic duct diameter vary considerably less, making these locations more appropriate for a single-size-fits-all device. A device may be placed in the infundibulum to prevent passage of gallstones from the fundus and body into the neck and cystic duct. The device may be designed to account for the dramatic taper of the infundibulum as it narrows towards the neck and the contractile force of the infundibular wall.

FIGS. 11A and 11B illustrate the gallbladder 10 as shown in FIG. 1A in an expanded and contracted condition, respectively, with a section of the gallbladder wall 8 removed to show an exemplary gallbladder treatment device 100. In this embodiment, the device 100 has a rounded 126 frame contour 125 within the gallbladder infunibulum 16 interior. The device 100 is positioned in an orientation to maintain the gallstones or gallstone fragments 9 on one side of the device 100 and away from the gallbladder neck 18.

FIG. 11A illustrates a device 100 located in the infundibulum. Devices constructed from porous flexible materials, rigid frames, or a combination of the two as described above can be modified for use in the infundibulum 16. Modification includes changing the diameter of the device to fit the smaller and more uniformly circular cross-section of the infundibulum, for example 1.5-2 cm in diameter and 0.5-2 cm in length, while retaining the ability to change shape during gallbladder contraction.

FIG. 11B illustrates the device 100 in a collapsed configuration within the infundibulum 16 of a contracted gallbladder.

FIGS. 11C and 11D illustrate the gallbladder as shown in FIG. 1A in an expanded and contracted condition, respectively, with a section of the gallbladder wall 8 removed to show an exemplary gallbladder treatment device 100. In this embodiment, a conical 127 frame contour 125 within the gallbladder infunibulum 16 interior. The device 100 is positioned in an orientation to maintain the gallstones or gallstone fragments 9 on one side of the device 100 and away from the gallbladder neck 18.

Further modification of a gallbladder treatment device to suit the anatomy of the infundibulum 16 may include shortening the anchoring struts and reducing the amount of flexion permitted at certain points, for example where the device meets the gallbladder wall. Struts 120 or anchoring members 142 may be shaped or angled to conform to the tapered geometry of the infundibulum. Because contraction is less dynamic in the infundibulum than the fundus or body, circumferential struts or rings may be employed to anchor and stabilize the device without exerting enough radial force to penetrate or erode through the wall of the organ.

The neck 18 is a narrow, S-shaped area in the most proximal part of the gallbladder and contains the outlet to the cystic duct 26, beyond which is the spiral valve of Heister 24. A device may be placed in the neck 18 or cystic duct 26 in order to prevent the passage of gallstones 9 into the spiral valve of Heister and the common bile duct. Any of the anchored designs described above for use in other parts of the gallbladder may be modified for use in the neck 18.

FIGS. 12A and 12B illustrate the gallbladder 10 as shown in FIG. 1A in an expanded and condition with a section of the gallbladder wall 8 removed to show an exemplary gallbladder treatment device 100 with a conical frame contour within the gallbladder neck or cystic duct entrance. The device 100 is positioned in an orientation to maintain the gallstones or gallstone fragments 9 on one side of the device 100 and unable to enter within or block the gallbladder neck 18 or cystic duct 26.

FIGS. 12A and 12B illustrate a gallbladder treatment device in the gallbladder neck (FIG. 12A) with an enlarged view of the device 100 and surrounding anatomy shown in FIG. 12B. FIG. 12A illustrates a gallbladder treatment device modified to fit into the neck of the gallbladder. The device is anchored inside the cystic duct. In this location, the device does not change shape or position with contraction/expansion cycles of the gallbladder since this portion of the gallbladder is substantially unchanged during gallbladder function. Modification of a design to suit the anatomy of the neck and cystic duct may include decreasing the cross-sectional area of the device to fit in the neck and elongating the filtering portion of the device to extend into the infundibulum. The materials and construction techniques described above as well as the fixation techniques described herein in relation to FIGS. 3, 3A, 3B, 3C, 3D, 8A, 8B and 8C may also be employed in a device placed in the neck or anchored in the cystic duct.

In light of the foregoing, there are provided herein a number of alternative gallbladder treatment devices for maintaining a gallstone or a gallstone fragment within a portion of a gallbladder distal to a gallbladder neck. In one aspect, the device includes a frame formed from at least one element having a filtering characteristic dimensioned to prevent the passage of a gallstone or gallstone fragment having a largest dimension of greater than 3 mm. There is also a plurality of struts extending from the frame to secure the frame in an orientation transverse to a gallbladder central, longitudinal axis. As a result, the gallstone or the gallstone fragments having a largest dimension of less than 3 mm remains within the gallbladder on one side of the frame. In one variation, the frame has a generally rounded contour with a radius of curvature between about 0.1 cm to about 1.0 cm. In another variation, the frame has a generally conical contour with a radius of curvature between about 0.1 cm to about 1.0 cm.

In still other embodiments, there is provided a gallbladder treatment device for maintaining a gallstone or a gallstone fragment within a portion of a gallbladder distal to a gallbladder neck. In this embodiment, there are a plurality of struts arranged in a generally cylindrical pattern and a wall contacting section on each of the plurality of struts. There is also a frame formed from a plurality of flexible members attached between two of the plurality of struts wherein the spacing between two adjacent flexible members when attached between two of the plurality of struts is less than 3 mm such that in use in the gallbladder the frame maintains the gallstone or the gallstone fragment within a portion of a gallbladder distal to a gallbladder neck. In addition, the radius of curvature formed by the plurality of flexible members is variable between a first curvature when the plurality of struts are contacting an expanded gallbladder and a second curvature when the plurality of struts are contacting a contracted gallbladder. In one aspect, a filter material is supported on the frame.

In still another alternative of a gallbladder treatment device for maintaining a gallstone or a gallstone fragment within a portion of a gallbladder distal to a gallbladder neck, the device includes a structure formed from at least one element having a filtering characteristic dimensioned to prevent the passage of a gallstone or gallstone fragment having a largest dimension of greater than 3 mm. There is also one or more fixation elements spaced along and attached to the perimeter of the structure.

In one aspect, the filtering structure comprises a braided structure, or a mesh filter of intersection strands. The strands may be made from a shape memory metal or be strands of a suture material. The structure may be a flexible sheet in one aspect. When formed from a sheet, the filtering characteristic dimensioned to prevent the passage of a gallstone or gallstone fragment having a largest dimension of greater than 3 mm are a plurality of perforations formed in the flexible sheet.

In another aspect of a gallbladder treatment device, the structure formed from at least one element having a filtering characteristic dimensioned to prevent gallstone passage is dimensioned to span a portion of the gallbladder transverse to the gallbladder central longitudinal axis. The portion of the gallbladder transverse to the gallbladder central longitudinal axis includes, for example, a portion of the gallbladder fundus; a portion of the gallbladder body; a portion of the gallbladder infundibulum; and a portion of the gallbladder neck.

### Device Delivery

FIG. 13A illustrates the gallbladder 10 as shown in FIG. 1A with a section of the gallbladder wall 8 removed to show an exemplary gallbladder treatment device 100. The device 100 is in a stowed condition at the distal end of a delivery device or catheter 270 that has been passed through an opening 295 in the gallbladder wall and into the gallbladder interior 11.

FIG. 13B illustrates the gallbladder and treatment device 100 as shown in FIG. 13A undergoing a balloon dilation procedure. this procedure is used to position the gallbladder treatment device 100 within the gallbladder body 11 in an orientation to maintain the gallstones or gallstone fragments 9 on one side of the device 100 and away from the gallbladder neck 18.

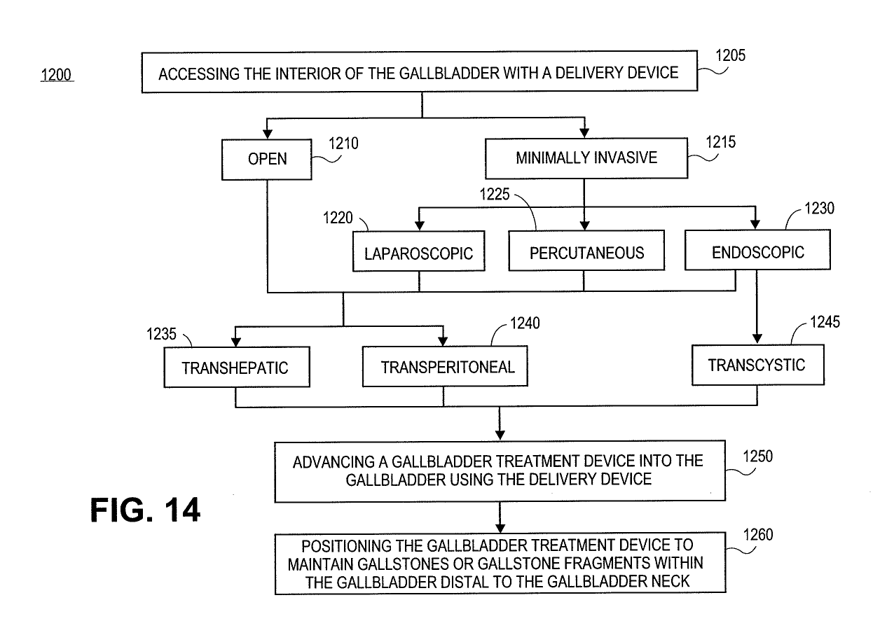

FIG. 14 is a block diagram illustrating various methods of accessing the interior of the gallbladder, advancing a gallbladder treatment device into the gallbladder interior and positioning the gallbladder treatment device within the gallbladder interior in an orientation to maintain the gallstones or gallstone fragments on one side of the device and away from or distal to the gallbladder neck.

Access to the gallbladder lumen can be obtained through open or minimally invasive surgical procedures. FIG. 14 is a chart illustrating various aspects of the inventive method 1200 of accessing, advancing and positioning a gallbladder treatment device within the gallbladder to maintain gallstones or gallstone fragments within the gallbladder distal to the gallbladder neck and one side of the gallbladder treatment device.

In general terms, FIG. 14 illustrates a method 1200 that includes the steps of accessing 1205, advancing 1250 and positioning 1260 a gallbladder therapy device to segregate the gallbladder thereby isolating gallstones and gallstone fragments from the gallbladder neck.

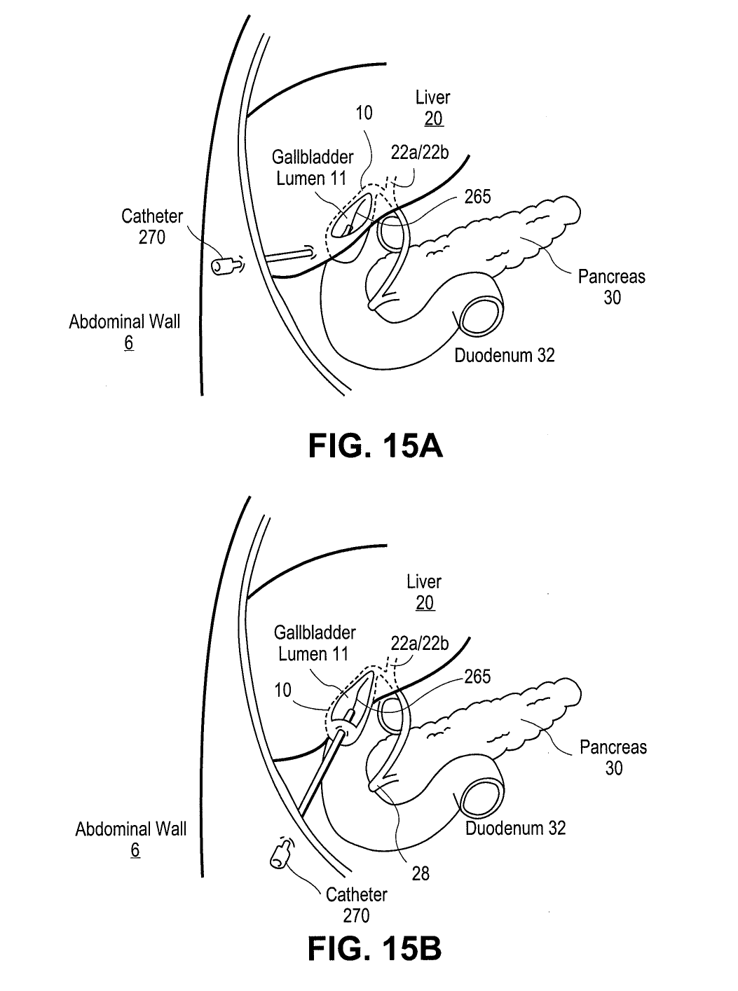

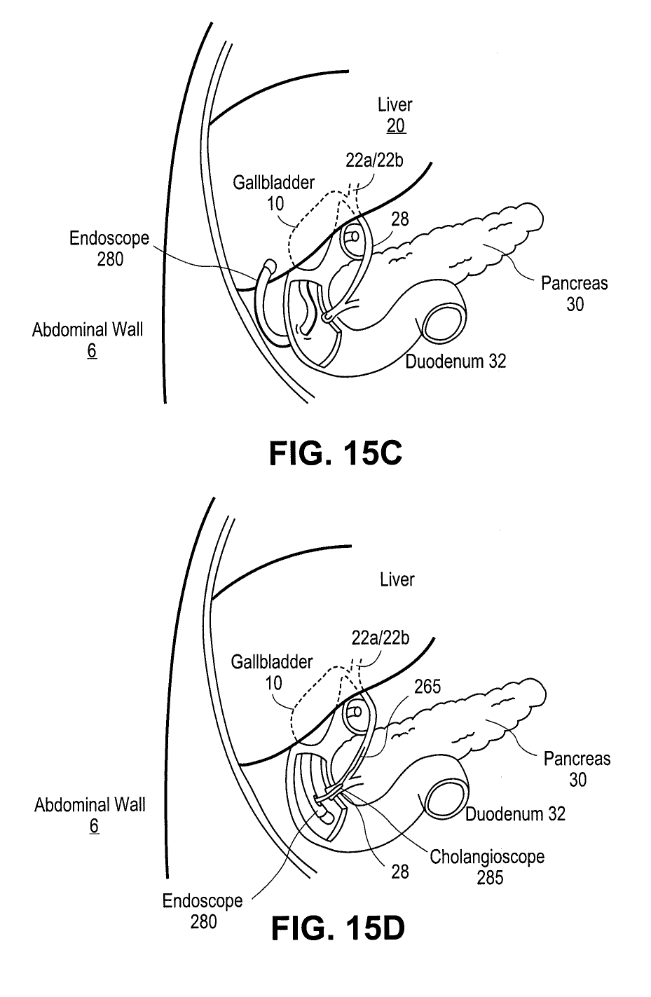

Step 1205 (accessing the gallbladder interior) may be accomplished using open 1210 or minimally invasive 1215 surgical techniques. Minimally invasive surgical techniques include laparoscopic techniques 1220, percutaneous techniques 1225, and endoscopic techniques 1230. Open surgery 1210, laparoscopic techniques 1220, percutaneous techniques 1225, and endoscopic techniques 1230 may be applied to both transhepatic 1235 and transperitoneal 1240 approaches. Endoscopic techniques 1230 may also be applied to transcystic approaches 1245. As set forth in further detail below, each of these basic steps 1205, 1250, 1260 and accessing alternatives (blocks 1210-1245) are subject to a number of variations and alternatives as further described below. Additionally, a number of specific implementations of the method 1200 are illustrated in FIGS. 15A-15D.

FIG. 15A illustrates the gallbladder within the abdomen and in relation to the liver, the pancreas and the duodenum with a section of the gallbladder wall removed to show accessing the gallbladder interior volume. A guide wire and a delivery catheter have entered the gallbladder using a transhepatic approach.

FIG. 15B illustrates the gallbladder 10 within the abdomen and in relation to the liver 20, the pancreas 30 and the duodenum 32. A section of the gallbladder wall 8 is removed to show accessing the gallbladder interior volume 11. A guide wire 265 and a delivery catheter 270 have entered the gallbladder using a transperitoneal approach.

FIG. 15C illustrates the gallbladder 10 within the abdomen and in relation to the liver 20, the pancreas 30 and the duodenum 32. This view illustrates accessing the gallbladder endoscopically by advancing an endoscope 280 through an opening in the duodenum and advancing towards the gallbladder 10.

FIG. 15D illustrates the gallbladder 10 within the abdomen and in relation to the liver 20, the pancreas 30 and the duodenum 32. This figures shows accessing the gallbladder endoscopically by advancing an endoscope 280 into the duodenum 32. Once in the duodenum 32, a guidewire 265 and then cholangioscope 285 are advanced into the common bile duct 28 and the advanced towards the gallbladder.

The gallbladder 10 can be visualized and accessed either through traditional open surgical techniques or using minimally invasive interventional methods. Delivery of a gallbladder treatment device may employ a delivery catheter 270 to advance, position and deploy the gallbladder treatment device 100 inside the gallbladder lumen 11. The delivery catheter 270 may be sized to work with other conventional catheter sets. For example, the delivery catheter may be 4-12 F in diameter. The delivery catheter may be radiopaque. The delivery catheter may follow a guidewire 265 into the gallbladder lumen (see, e.g., FIGS. 15A and 15B).

Open surgery provides direct visualization of the abdominal compartment. The gallbladder 10 is exposed by lifting inferior edge 20a of the liver 20 superiorly and retracting the gallbladder up and over the dome of the liver. Either a transhepatic or transperitoneal approach may be used to enter the gallbladder and deliver a device. The pros and cons of each approach are discussed below. The access site in the gallbladder (and liver) may be closed with suture, surgical glue, or a commercially available vascular closure device.

Minimally invasive techniques, including laparoscopic, percutaneous, or endoscopic can be used to access and deliver a device without general anesthesia or a large incision. These approaches rely on indirect methods of visualizing the anatomy. A laparoscopic approach to deliver a device to the gallbladder would begin with trocar placement, CO2 insufflation and camera introduction as performed in traditional laparoscopic surgery, for example cholecystectomy or appendectomy. The gallbladder is entered through either a transhepatic approach or transperitoneal approach as illustrated, respectively, in FIGS. 15A and 15B.

In a percutaneous approach, ultrasound or fluoroscopy are used to visualize abdominal organs. Under real-time imaging, a needle is passed through the skin in the right upper quadrant of the abdomen. Once through the abdominal wall, the needle is guided through the liver (transhepatic see FIG. 15A) or through the abdomen (transperitoneal see FIG. 15B) and into the gallbladder. Location inside the gallbladder is confirmed by contrast injection and visualization of the boundaries of the gallbladder lumen. A guidewire 265 is passed through the needle, which is then withdrawn, leaving the guidewire 265 in place. The gallbladder treatment device delivery catheter 270 is advanced over the guidewire 265 and into the gallbladder interior 11. Position is confirmed using real-time imaging with contrast injection. Once the gallbladder treatment device 100 is delivered, the delivery catheter 270 is withdrawn. The access tract may be closed using a commercially available vascular closure device, traditional suture or surgical glue as discussed in greater detail below.

An endoscopic approach involves delivering the gallbladder treatment device 100 through an endoscope 280, starting from a trans-oral, trans-rectal or trans-vaginal approach. Trans-oral and trans-rectal endoscopy can employ a transhepatic, transabdominal or transcystic (cholangioscopic in FIG. 15D) approach to deliver a gallbladder treatment device to the gallbadder. Both approaches require entering the abdominal cavity by passing the endoscope 280 through the wall of the GI tract. FIG. 15C illustrates such an approach through a wall of the duodenum 32. Trans-vaginal endoscopy is limited to transhepatic or transabdominal approaches.

Percutaneous access to the gallbladder has been described for aspiration, stone dissolution, and placement of biliary drains and stents. There are two common approaches: transhepatic (see FIG. 15A), where the entry needle and guidewire are passed through the liver en route to the gallbladder, and transperitoneal (see FIG. 15B), where the free wall of the gallbladder is accessed from the abdominal cavity. Both approaches are performed under local anesthesia with ultrasound or fluoroscopic guidance.

Transhepatic access is more common and generally considered to have fewer complications than transperitoneal since the adjacent liver will act to tamponade any bile leaks from the puncture site in the gallbladder wall. Transhepatic access is aimed at entering the liver through the superior “bare area”, where there is no peritoneal covering. The Seldinger technique is employed, where an 18 to 22 gauge entry needle, for example a Chiba needle, is inserted into the gallbladder followed by a flexible hydrophillic guidewire passed through the needle lumen into the gallbladder. A small amount of radiocontrast may be injected into the gallbladder through the entry needle to confirm location. The access passage can be dilated safely to accommodate an 8 to 10 F catheter.

Tranperitoneal access is sometimes preferred in the presence of severe liver disease or coagulopathy in order to prevent hemorrhage or infectious complications. However, bile leakage from the puncture site into the peritoneum is a common complication. Long term (4-6 week) drain placement is often necessary to promote tract formation and epitheliazation through the puncture site and avoid internal bile leakage. Bile leakage can be prevented and drain placement avoided by using a commercially available vascular closure device to seal the access tract. These devices are mentioned and a specific implementation is discussed below. The guidewire and imaging components of transperitoneal access are identical to those used in the transhepatic approach.

Either the transhepatic or transperitoneal approach can be modified to deliver a gallbladder treatment device to the body 14, fundus 12, infundibulum 16 or neck 18 of the gallbladder 10. For the purposes of delivering a treatment device the two approaches are considered medically equivocal once access to the gallbladder lumen is confirmed. After obtaining access to the gallbladder, a guidewire with a radiolucent or echogenic tip may be passed and anchored in specific positions corresponding to the intended location of the gallbladder treatment device.

For example, to deliver a device to the fundus or body, the guidewire can be advanced and anchored in the distal ⅓ of the gallbladder, in the central body. For delivering a device to the infundibulum or neck, the guidewire can be advanced into the proximal ⅓ of the organ and anchored. For delivering a device to the cystic duct or proximal neck, the guidewire may be advanced up to or past the spiral valve of Heister.

Once the guidewire 265 is in position, a delivery catheter 270 may be advanced over the guidewire 265 which contains the therapy device 100 in an unexpanded or stowed configuration (see FIG. 13A). The gallbladder treatment device 100 is advanced through the delivery catheter 270 and into final position over the exposed guidewire 265. The device may self-expand into place after it exits the delivery catheter. Alternatively, the gallbladder therapy device may be triggered to expand by the operator, or released from a cartridge or case.

Additionally or alternatively, a balloon catheter dilation technique may be used within the gallbladder lumen to in stabilization of the delivery catheter, confirmation of device position within the gallbladder and aid in device deployment and engagement. FIG. 13B illustrates the use of a balloon 260 within a device 100 expanded to engage the device with the gallbladder wall.

In one alternative, the gallbladder therapy device delivery method (FIG. 14) includes the use of balloon dilation to locate the intersection of the highly elastic fundus and body and the less elastic infundibulum. Based on the result of the balloon dilation procedures, the guidewire and delivery catheter can be repositioned accordingly. In still other balloon dilation embodiments, a balloon dilation technique may be used to deploy the device, to ensure apposition with the gallbladder wall, ands/or to engage fixation elements and the like. In still other embodiments, a balloon dilation technique may also be used after delivery of the device to promote adhesion or anchoring of the device against the gallbladder wall at any location. In this technique, after deploying the gallbladder treatment device into the gallbladder, suction is applied to the gallbladder lumen. As the gallbladder lumen is evacuated, the gallbladder is drawn down onto the device and into solid apposition with any fixation device or structure to ensure the device remains in the delivery position and orientation.

FIG. 15D illustrates the gallbladder within the abdomen and in relation to the liver 20, the pancreas 30 and the duodenum 32 showing accessing the gallbladder endoscopically by advancing an endoscope 280 into the duodenum 32 and then advancing a guidewire 265 and cholangioscope 285 into the common bile duct 28 and advancing towards the gallbladder. FIG. 15D will be used to discuss an alternative method of accessing the gallbladder using gastrointestinal endoscopy. In this approach, a traditional perioral endoscope is used to access the common bile duct followed by deployment of a smaller cholangioscopic device through the working channel of the parent scope. The cholangioscope 285 is advanced in a retrograde fashion up the common and cystic ducts and into the gallbladder lumen. Once access to the gallbladder lumen is confirmed, guidewire positioning and catheter delivery methods described above, including balloon dilation (e.g., FIG. 13B), can be used to accurately place the gallbladder treatment device at a specific site within the cystic duct, neck, infundibulum or body and fundus of the gallbladder.

In consideration of the foregoing, there are provided herein a number of methods for maintaining a gallstone or a gallstone fragment within a portion of a gallbladder distal to a gallbladder neck. In some embodiments, the methods include accessing the interior of the gallbladder with a delivery device. Next, there is the step of advancing a gallbladder treatment device into the gallbladder interior using the delivery device. Thereafter, the gallbladder treatment device is positioned within the gallbladder to maintain a gallstone or a gallstone fragment within a portion of the gallbladder distal to the gallbladder neck and one side of the gallbladder treatment device.

Accessing the interior of the gallbladder may be accomplished with an open surgical procedure or one of a variety of minimally invasive surgical (M.I.S.) procedures.

The minimally invasive surgical procedure includes, for example, accessing the interior of the gallbladder with an endoscopic or a laparoscopic approach technique. Moreover, the minimally invasive surgical procedure includes, for example, accessing the interior of the gallbladder with a percutaneous or a transluminal approach technique. In addition, both open and M.I.S. procedures may include accessing the interior of the gallbladder with a transhepatic or a transperitoneal approach technique.

In another aspect of the inventive methods, the advancing step includes, for example, passing the gallbladder treatment device in a stowed condition through at least a portion of a liver including, for example, a portion of the liver lacking a peritoneal covering. In still another aspect of the inventive methods, the advancing step includes, for example, passing the gallbladder treatment device in a stowed condition through at least a portion of a peritoneal cavity or through at least a portion of the gastrointestinal tract. Still further, in some aspects of passing the gallbladder treatment device through at least a portion of the gastrointestinal tract the stowed gallbladder treatment device is passed though a portion of an esophagus, a stomach, a duodenum, a small intestine, or a colon. In still other aspects, alternatives of the advancing step include, for example, passing the gallbladder treatment device through a wall of the gastrointestinal tract. In still other embodiments, the step of passing through a wall of the gastrointestinal tract includes a wall in a portion of an esophagus, a stomach, a duodenum, a small intestine, or a colon.

In still other aspects of the inventive methods, the advancing step includes, for example, passing the gallbladder treatment device in a stowed condition through a portion of a fundus of the gallbladder; through a portion of a body of the gallbladder; through a portion of an infundibulum of the gallbladder; through an generally inferior aspect of the gallbladder; through a generally superior aspect of the gallbladder; and/or through a generally lateral aspect of the gallbladder.

In still additional aspects, the positioning step includes, for example, adding and/or removing fluid from the gallbladder. In another aspect, the positioning step includes, for example, activating a delivery device. In one version, activating a delivery device includes inflating a balloon. In another variation, the positioning step includes, for example, moving or transitioning the gallbladder treatment device from a stowed configuration to a deployed configuration. Still further, the positioning step may include adjusting the orientation of the gallbladder treatment device within the gallbladder.

In one aspect, after the positioning step, the gallbladder treatment device is positioned within the gallbladder in an orientation that retains a portion the gallstones and stone fragments in the gallbladder substantially within a fundus of the gallbladder. In another variation, after the positioning step, the gallbladder treatment device is positioned within the gallbladder in an orientation that retains a portion the gallstones and stone fragments in the gallbladder substantially within a fundus and a body of the gallbladder. In another variation, after the positioning step, the gallbladder treatment device is positioned within the gallbladder in an orientation that retains a portion the gallstones and stone fragments in the gallbladder substantially within a fundus, a body and a portion of an infundibulum of the gallbladder. Moreover, it is appreciated that, after the positioning step, the gallbladder treatment device is against a wall of a fundus of the gallbladder; against a wall of a body of the gallbladder; against a wall of an infundibulum of the gallbladder or against a less elastic portion of a wall of an infundibulum of the gallbladder.

The various methods described above are subject to further alternatives. Any of the methods above may include the additional step of anchoring the gallbladder treatment device to the gallbladder wall or a portion thereof. Still further, the methods may also include the step inflating the gallbladder after the accessing step and before the advancing and positioning steps. Similarly, any of the methods above may also include collapsing the gallbladder around the gallbladder delivery device after the advancing or the positioning steps. Collapsing may be accomplished using suction from a catheter delivery device, endoscope, or other acceptable surgical device and suction procedure.

Device positioning can be further enhanced by injecting and removing contrast to inflate/deflate the organ under fluoroscopic imaging. Closure of the gallbladder and transhepatic access tract (if used) can be achieved using a number of currently available vascular closure devices, for example Angio-Seal, Perclose, Duett Pro, Vaso-Seal, X-Site, Super Stitch, Angiolink EVS and Starclose.

In one illustrative embodiment, a puncture or incision 295 formed in the gallbladder to introduce the gallbladder treatment device is sealed with a closure device and method similar to those described in U.S. Pat. No. 5,021,059 titled “Plug Device With Pulley for Sealing Punctures In Tissue and Methods of Use,” to Kenneth Kensey, et al. (“Kensey”). The method and devices described by Kensey are modified to accommodate use in the gallbladder as described herein. The closure device is provided through the access created during the accessing step. A portion of the closure device is secured within the gallbladder adjacent to the wall opened during the accessing step. While pressure is applied to the gallbladder wall a suitable volume of a filler material is inserted into the opening and access pathway sufficient to close the opening in the gallbladder wall and the access pathway including any opening in another organ, such as a portion of the liver. The volume of fill material used may range from 20-30 cc or more depending upon the specific patient conditions, access pathway length and dimensions. In one aspect the volume of fill material used is between 20 to 50 cc, or between 30-40 cc. Suitable filler materials include collagen, hydrogel, fibrin glue, and the like or combinations thereof.

As for additional details pertinent to the present invention, materials and manufacturing techniques may be employed as within the level of those with skill in the relevant art. The same may hold true with respect to method-based aspects of the invention in terms of additional acts commonly or logically employed.

As such, numerous additional alternative embodiments are also included. For example, the cross section shape of the elements of the gallbladder treatment device (i.e., the frame, the struts or the elements of a filtering embodiment or structure) may be any one or a combination of circular, oval, oblong, D-shaped, C-shaped, U-shaped, or rectangular. In much the same way, it is to be appreciated that the radial force and strut length variations shown and described in FIGS. 3A, 11C, 11D, 5A and 5B as well as in the various fastening and fixation teachings shown and described in relation to FIGS. 3B, 3C and 3D are not limited only to those described exemplary embodiments. These various aspects of the gallbladder treatment device design and fixation may be applied to the other alternative gallbladder treatment device embodiments described herein.

Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

It is intended that the following claims define the scope of the invention and that methods and structures within the scope of these claims and their equivalents be covered thereby.

1. A gallbladder treatment device for maintaining a gallstone or a gallstone fragment within a portion of a gallbladder distal to a gallbladder neck, comprising:
A frame formed from at least one element having a filtering characteristic dimensioned to prevent the passage of a gallstone or gallstone fragment having a largest dimension of greater than 3 mm; and
A plurality of struts extending from the frame to secure the frame in an orientation transverse to a gallbladder central, longitudinal axis such that the gallstone or the gallstone fragment having a largest dimension of less than 3 mm remains within the gallbladder on one side of the frame.

2. The gallbladder treatment device of claim 1 wherein the frame has a generally rounded contour with a radius of curvature between about 0.1 cm to about 1.0 cm.

3. The gallbladder treatment device of claim 1 wherein the frame has a generally conical contour with a radius of curvature between about 0.1 cm to about 1.0 cm.

4. The gallbladder treatment device of claim 1 wherein the at least one element is a flexible sheet of material and the filtering characteristic is a plurality of apertures formed in the flexible sheet having a largest dimension of less than 3 mm.

5. The gallbladder treatment device of claim 1 wherein the at least one element is a plurality of flexible strands from a braided structure or a plurality of suture strands that maintains a filtering characteristic that is the spacing between adjacent strands that is less than 3 mm.

6. (canceled)

7. The gallbladder treatment device of claim 1 wherein the plurality of struts are shaped to deflect outwardly against the gallbladder wall with a radial force sufficient to maintain the frame in the orientation transverse to a gallbladder central, longitudinal axis.

8. (canceled)

9. The gallbladder treatment device of claim 1 wherein a portion of the struts comprises a fixation element to engage with a wall of the gallbladder.

10. The gallbladder treatment device of claim 9 wherein the fixation element is a plurality of spikes.

11.-17. (canceled)

18. The gallbladder treatment device of claim 1 wherein at least a portion of the frame or a portion of the struts is coated with a material to inhibit gallbladder biofilm formation.

19. The gallbladder treatment device of claim 18 wherein the material is an antibiotic selected from the group of antibiotics suited to treat infections of the gastrointestinal tract.

20. (canceled)

21. The gallbladder treatment device of claim 1 wherein at least a portion of the frame or a portion of the struts is formed from materials having substantially non-porous, hydrophillic surfaces.

22. The gallbladder treatment device of claim 1 wherein the plurality of struts are shaped to conform to a portion of the curvature of a wall of the gallbladder fundus so as to maintain the frame in a portion of the gallbladder fundus in an orientation transverse to a gallbladder central, longitudinal axis in order to maintain the gallstone or the gallstone fragment having a largest dimension of greater than 3 mm on one side of the frame and within a more distal portion of the gallbladder fundus.

23. The gallbladder treatment device of claim 1 wherein the plurality of struts are shaped to conform to a portion of the curvature of a wall of the gallbladder body so as to maintain the frame in portion of the gallbladder body in an orientation transverse to a gallbladder central, longitudinal axis in order to maintain the gallstone or the gallstone fragment having a largest dimension of greater than 3 mm on one side of the frame and within a portion of the gallbladder fundus and a more distal portion of the gallbladder body.

24.-25. (canceled)

26. The gallbladder treatment device of claim 1 wherein the plurality of struts are shaped into a closed or substantially closed three dimensional structure or a sphere or an ovoid that is dimensioned to fit within the gallbladder interior.

27.-53. (canceled)

54. A gallbladder treatment device according to claim 9 further comprising: one or more fixation hook elements on the frame or the plurality of struts.

55.-60. (canceled)

61. A method of maintaining a gallstone or a gallstone fragment within a portion of a gallbladder distal to a gallbladder outlet, comprising:
Accessing the interior of the gallbladder with a delivery device;
Advancing a gallbladder treatment device into the gallbladder interior using the delivery device; and
Positioning and anchoring the gallbladder treatment device within the gallbladder to maintain a gallstone or a gallstone fragment within a portion of the gallbladder distal to the gallbladder outlet and one side of the gallbladder treatment device.

62. (canceled)

63. The method of claim 61 wherein the accessing step further comprising: accessing the interior of the gallbladder with a minimally invasive surgical procedure from one or more of: an endoscopic procedure, a laparoscopic procedure or a percutaneous procedure.

64.-65. (canceled)

66. The method of claim 61 the accessing step further comprising: accessing the interior of the gallbladder with a transhepatic approach or a transperitoneal approach technique.

67.-79. (canceled)

80. The method of claim 61 the positioning step further comprising adding and/or removing fluid from the gallbladder.

81. The method of claim 61 the positioning step further comprising: activating the delivery device by inflating a balloon within the gallbladder.

82.-87. (canceled)

88. The method of claim 61 wherein after the positioning and anchoring step a portion of the gallbladder treatment device is against a wall of the gallbladder.

89.-91. (canceled)

92. The method of claim 61 the positioning and anchoring step further comprising: reversibly positioning and anchoring the gallbladder treatment device within the gallbladder.

93.-94. (canceled)
